(19) United States
(12) Patent Application Publication
(10) Pub. No.: US 2026/0345246 A1
(43) Pub. Date: Nov. 5, 2026

(54) Alert System Behavior Based On Localization Awareness

(71) Applicant: Stryker Corporation, Portage, MI (US)

(72) Inventors: James G. Walen, Portage, MI (US); Zachary S. Bolthouse, Kalamazoo, MI (US); Lars Marius Schwalbe, Bollschweil (DE)

(73) Assignee: Stryker Corporation, Portage, MI (US)

(21) Appl. No.: 18/869,791

(22) PCT Filed: May 26, 2023
(86) PCT No.: PCT/US2023/023740

§ 371 Date: Nov. 26, 2024

Related U.S. Application Data
(60) Provisional application No. 63/346,075, filed on May 26, 2022.
(60) Provisional application No. 63/350,224, filed on Jun. 8, 2022.

(51) Int. Cl. A61B 34/20; A61B 17/00; A61B 17/16; A61B 34/10

(52) U.S. Cl. CPC A61B 34/20; A61B 17/1622; A61B 17/1626; A61B 34/10; A61B 2017/00119; A61B 2017/00199; A61B 2034/107; A61B 2034/2048

(57) ABSTRACT

A hand-held surgical system includes an instrument configured to be held in a hand of a user and including a drive motor. The instrument is coupled to a tracking device. The hand-held surgical system also includes a tracking unit, an alert module, and a control system in communication with the tracking unit, the alert module, and the instrument. The control system is configured to track a pose of the instrument in a known coordinate system with the tracking unit, determine a boundary associated with a region of interest in a surgical procedure in the known coordinate system, control the alert module based on the boundary and the tracked pose of the instrument, determine that an occlusion event has occurred for the tracking device, and control the drive motor based on the occlusion event, the boundary, and the tracked pose of the instrument.

### CROSS-REFERENCE TO RELATED APPLICATIONS

This application claims the benefit of and priority to U.S. Provisional Patent Application No. 63/346,075, filed May 26, 2022, and U.S. Provisional Patent Application No. 63/350,224, filed Jun. 8, 2022, the disclosure of each of which is incorporated herein by reference in its entirety.

### BACKGROUND

In modern surgery, medical personnel frequently utilize powered surgical instruments, such as cordless drills, saws, wire drivers, high speed drills, ultrasonic handpieces, and the like. Often these surgical instruments comprise a drive motor and/or processor within a handpiece or housing. The surgical instrument may comprise an attachment feature configured to receive a cutting attachment designed for application to a surgical site to perform a specific medical procedure. For example, a surgical drill may utilize a cutting attachment such as a drill bit, bur, or reamer for cutting bores into tissue or for selectively removing tissue such as bone. The ability to use powered surgical instruments on a patient lessens the physical strain of surgeons when performing medical procedures on the patient. Moreover, most surgical procedures can be performed more quickly and more accurately with powered surgical instruments than with the manual equivalents that preceded them.

In order to further increase the accuracy and efficacy of the surgical procedures involving powered surgical instruments, these instruments are often tracked by a localization system. These localization systems are capable of alerting the surgeon if the powered surgical instrument veers off course or moves close to a critical structure, such as a spinal cord. These localization systems often rely on a line-of-sight with the surgical instrument. Depending on the procedure being performed by the surgeon, the line-of-sight between the localization system and the surgical instrument may be frequently occluded. These occlusions may be especially frequent where the surgical instrument is a hand-held instrument that may be moved to any number of locations by the surgeon.

Although some alerts in response to loss of line-of-sight may be valuable to the surgeon, others may be less so, such as if the loss is inconsequential and/or insignificant. These latter alerts may be annoying or confusing to the surgeon. Further, if a system were to be configured to halt the operation of the surgical tool, the surgeon may find the tool that they are using frequently disabled in response to inconsequential and/or insignificant losses of line-of-sight.

As such, there is a need in the art for surgical systems and methods that can determine and distinguish between relatively significant and insignificant losses of line-of-sight to reduce the number of alerts and interruptions during a procedure.

### SUMMARY

According to a first aspect, a hand-held surgical system is provided, the system including: an instrument configured to be held in a hand of a user and including a drive motor, a console coupled to the instrument, a tracking device coupled to the instrument, a tracking unit, an alert module, and a control system in communication with the tracking unit, the alert module, and the console. The control system is configured to determine a pose of the instrument, determine a boundary, control the alert module based on the boundary and the pose of the instrument, determine that an occlusion event has occurred for the tracking device, and control the drive motor based on the occlusion event, the boundary, and the pose of the instrument.

According to a second aspect, a hand-held surgical system is provided, the system including: an instrument configured to be held in a hand of a user and including a drive motor, a tracking device coupled to the instrument, a tracking unit, an alert module, and a control system in communication with the tracking unit and the alert module. The control system is configured to determine a pose of the instrument, determine a boundary, control the alert module based on the boundary and the pose of the instrument, determine that an occlusion event has occurred for the tracking device, determine a duration of the occlusion event, determine a distance between the instrument and the boundary, and control the drive motor based on the occlusion event, the duration of the occlusion event, the boundary, the pose of the instrument, and the distance between the instrument and the boundary.

Any of the above aspects can be combined in part or in whole with any other aspect. Any of the above aspects, whether combined in part or in whole, can be further combined with any of the following implementations, in full or in part.

In some implementations, the control system is further configured to determine at least one of a duration of the occlusion event, a duration threshold, a distance between the instrument and the boundary based on the boundary and the pose of the instrument, a direction of movement of the instrument based on the pose of the instrument and the boundary, a velocity of the instrument based on the boundary and the pose of the instrument, and a motion parameter of the instrument based on poses of the instrument and boundaries. In some implementations, the motion parameter is selected from at least one of a direction of the instrument, a velocity of the instrument, a speed of the instrument, and an acceleration of the instrument.

In some implementations, the control system is further configured to control the drive motor based on at least one of: the duration of the occlusion event; the distance and the occlusion event; the duration, the distance, and the occlusion event; the duration and the duration threshold; the direction of movement of the instrument and the occlusion event; and the velocity of the instrument.

In some implementations, the control system is further configured to determine a first pose of the instrument based on the pose of the instrument at a first time, and determine a second pose of the instrument based on the pose of the instrument at a second time. In some implementations, the control system is further configured to determine the direction of the instrument based on the first pose of the instrument and the second pose of the instrument, and/or determine the velocity of the instrument based on the first pose of the instrument and the second pose of the instrument.

In some implementations, the system further comprises a localization awareness indicator in communication with the control system. In some implementations, the control system controls the localization awareness indicator based on the occlusion event. In some implementations, the localization awareness indicator is defined as at least one of an auditory, visual, and tactile indicator. In some implementations, the localization awareness indicator is defined as a software routine.

In some implementations, the alert module is at least one of a vibratable footswitch, a graphical user interface, a speaker, and a software routine. In some implementations, the software routine is configured to set the drive speed of the motor and/or stop the drive motor.

In some implementations, the system further comprising a switch in communication with the instrument and the control system, the switch configured to generate an input signal sufficient to control the instrument. In some implementations the control system is further configured to reactivate the drive motor based on the input signal.

In some implementations, the instrument further comprises a battery in electrical communication with the drive motor, and controlling the drive motor includes controlling the battery.

In some implementations, the boundary is a mesh. In some implementations, the mesh is derived from at least one of a patient image, and a segmentation of the patient image.

In some implementations, the system further comprises a patient tracker coupled to a patient, and the control system is further configured to determine a pose of at least a portion of the patient, and determine that the occlusion event has occurred for the patient tracker.

These and other configurations, features, and advantages of the present disclosure will be apparent to those skilled in the art. The present disclosure is not intended to be limited to or by these configurations, embodiments, features, and/or advantages.

### BRIEF DESCRIPTION OF THE DRAWINGS

Advantages of the present invention will be readily appreciated as the same becomes better understood by reference to the following detailed description when considered in connection with the accompanying drawings, wherein:

FIG. 1 is a schematic view of a surgical system including a plurality of surgical instrument assemblies and a surgical navigation system for tracking a surgical instrument associated with each of the various surgical instrument assemblies.

FIG. 2 is a perspective view of an exemplary layout of an operating room including at least one of the surgical instrument assemblies and the surgical navigation system of FIG. 1 for performing a medical procedure on a patient.

FIG. 3 is a block diagram of a surgical system.

FIG. 4 is a schematic view of a first exemplary surgical instrument of the surgical system of FIG. 1, the first surgical instrument oriented in an exemplary position relative to the patient.

FIG. 5 is a schematic view of a second exemplary surgical instrument of the surgical system of FIG. 1, the second surgical instrument oriented in an exemplary position relative to the patient and a defined set of exemplary alert zones.

FIG. 6 is a schematic of an exemplary surgical system including a navigation system and a hand-held surgical instrument, the hand-held surgical instrument comprising a battery module and a plurality of interchangeable end-effectors.

FIG. 7 is a schematic of an exemplary surgical system including a navigation system and a high-speed bur, the high-speed bur comprising a control console and a plurality of interchangeable cutting burs.

FIG. 8 is a schematic view of an exemplary graphical user interface (GUI) of a navigation system including an alert interface.

FIG. 9 is a schematic view of an exemplary graphical user interface (GUI) of a navigation system including an occlusion event interface and a loss-of-localization-awareness indicator.

### DETAILED DESCRIPTION

During modern surgical procedures, tracking information may be used to verify whether surgical instruments are following planned surgical pathways and/or maintaining a safe distance away from critical anatomical structures, and correspondingly to provide feedback and/or notify the medical professional executing the procedure when the surgical instrument becomes misaligned with the surgical pathway and/or is at risk of impinging on a critical anatomical structure. To this end, it is also beneficial to provide feedback and/or notify medical professionals when/if the tracking becomes unreliable such that the tracking information is no longer able to determine whether the surgical instruments are following planned surgical pathways and/or maintaining a safe distance away from critical anatomical structures.

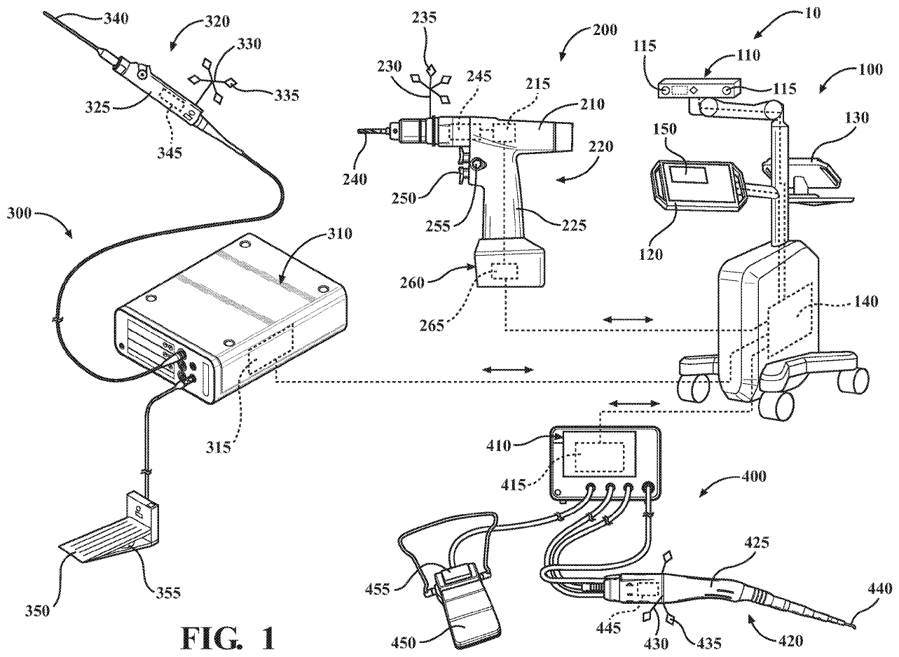

Accordingly, FIG. 1 illustrates an exemplary surgical system 10 that may include a surgical navigation system 100 for tracking one or more surgical instrument assemblies 200, 300, 400 including respective surgical instruments 220, 320, 420 to assist the medical professional, such as a surgeon, in executing a medical procedure.

The surgical navigation system 100 may include a navigation interface that includes one or more display units 120 and one or more user input devices 130, which may be realized as touch screen mechanisms integrated with the display units 120. The display units 120 of the surgical navigation system 100 may be configured to display one or more graphical user interfaces (GUI) 150, which may include various prompts or data entry boxes for receiving user input. For example, the display unit 120 may be configured to display a text box or prompt that allows the surgeon to manually enter or select the type of surgical procedure to be performed. The display unit 120 may also be configured to display patient data, such as a pre-operative image or scan. The pre-operative image may be based on MRI scans, radiological scans or computed tomography (CT) scans of the patient's anatomy. The preoperative image may be uploaded to the surgical navigation system 100 and displayed on the display unit 120. The display unit 120 may be further configured to display a surgical plan for a medical procedure overlaid on the patient data or image.

The surgical plan may include the surgical pathway for executing the medical procedure, and/or the planned trajectory and/or orientation for the medical instrument during the medical procedure. The surgical plan may also include the position and/or orientation of an implant or medical device to be inserted into a region of interest during the medical procedure, overlayed on the patient data or image. It is contemplated that the surgical navigation system 100 may include a display unit 120 configured to display and/or project a holographic image of the surgical pathway for executing the medical procedure, and/or the planned trajectory and/or orientation for the medical instrument during the medical procedure. This may include projecting the surgical pathway onto the patient or other surface in the operating room. It may also include a projection of the surgical pathway onto a head unit worn by the surgeon, such as a lens, shield, or glasses of the head unit. An exemplary configuration of surgical navigation system 100 including a display unit worn by the surgeon to display a target trajectory and/or target location of a medical procedure is disclosed in International Patent Application No. PCT/IB2018/053130, the entirety of which is hereby incorporated by reference.

The user input device(s) 130 and/or the graphical user interface (GUI) 150 may be configured to allow the surgeon to input or enter patient data and/or modify the surgical plan. The patient data may include patient images, such as pre-operative images of the patient's anatomy. These images may be based on MRI scans, radiological scans or computed tomography (CT) scans of the patient's anatomy. The patient data may also include additional information related to the type of medical procedure being performed, the patient's anatomical features, the patient's specific medical condition, and/or operating settings for the surgical navigation settings. For example, in performing a spinal surgery, the surgeon may enter information via the user input device(s) 130 and/or the graphical user interface (GUI) 150 related to the specific vertebra on which the medical procedure is being performed. The surgeon may also input various anatomical dimensions related to the vertebrae and/or the size and shape of a medical device or implant to be inserted during the medical procedure. The user input device(s) 130 and/or the graphical user interface (GUI) 150 may also be configured to allow the surgeon to select, edit or manipulate the patient data. For example, the surgeon may identify and/or select anatomical features from the patient data that define regions of interest to the medical procedure. This may include selecting one or more surgical sites, such as selecting one or more vertebrae and/or specific areas on the one or more vertebrae, where the medical procedure is to be performed.

The surgeon may also be able to identify critical anatomical features defining regions of interest to the medical procedure, such as anatomical features that the surgeon may want to either target or avoid during the medical procedure. For example, the surgeon may use the user input device(s) 130 and/or the graphical user interface (GUI) 150 to select cortical walls or portions thereof, nerves, blood vessels or similar critical anatomical structures that the surgeon wishes to avoid and establish zones surrounding those anatomical structures. The surgeon may also use the user input device(s) 130 and/or the graphical user interface (GUI) 150 to select and/or input a target location, target area to be resected, target trajectory, target depth or similar feature of the surgical pathway that is associated with a region of interest to help guide the surgeon in performing the medical procedure.

The system may be configured to utilize segmentation to facilitate identification of zones and/or boundaries associated with regions of interest to a medical procedure. This segmentation may be performed automatically, semi-automatically, or manually.

In one example of manual segmentation, the surgeon may utilize the user input device(s) 130 and/or the graphical user interface (GUI) 150 to define a geometric primitive associated with a region of interest to the medical procedure. A method of defining geometric primitives for the purpose of segmentation and visualization of cavities or orifices of the human body may include the steps of: manual pre-segmentation by defining enclosing geometric primitives in a 3D patient image for generating initial envelopes; analyzing the anatomy within pre-segmented geometric primitives; using the result of the analysis for adjustment of the envelopes; and visualizing the envelopes. The adjustment of a visualized envelope can be based on analyzed anatomy using computed voxel affiliations and the adjustment of a visualized cell envelope may be achieved by computing a surface mesh of the voxels which are affiliated completely and/or partially to the cell. Further, the adjustment of a visualized envelope may be achieved by optimizing type, orientation, position and/or size of the enclosing geometric primitive. Exemplary methods and systems for defining a geometric primitive and guiding a surgical instrument are disclosed in U.S. patent application Ser. No. 15/300,414 and U.S. patent application Ser. No. 15/582,637, both of which are hereby incorporated by reference in their entirety.

The user input device(s) 130 and/or the graphical user interface (GUI) 150 may also be configured to enable input of the surgical plan. This may include selecting the surgical instrument to be used and a device and/or implant to be inserted. It may also include identifying a position and/or orientation (i.e., pose) where the device or implant is to be placed within the patient as a region of interest. The user input device(s) 130 and/or the graphical user interface (GUI) 150 may also allow the surgeon to select the parameters of the implant to be inserted, such as the length and/or diameter of a screw to be inserted.

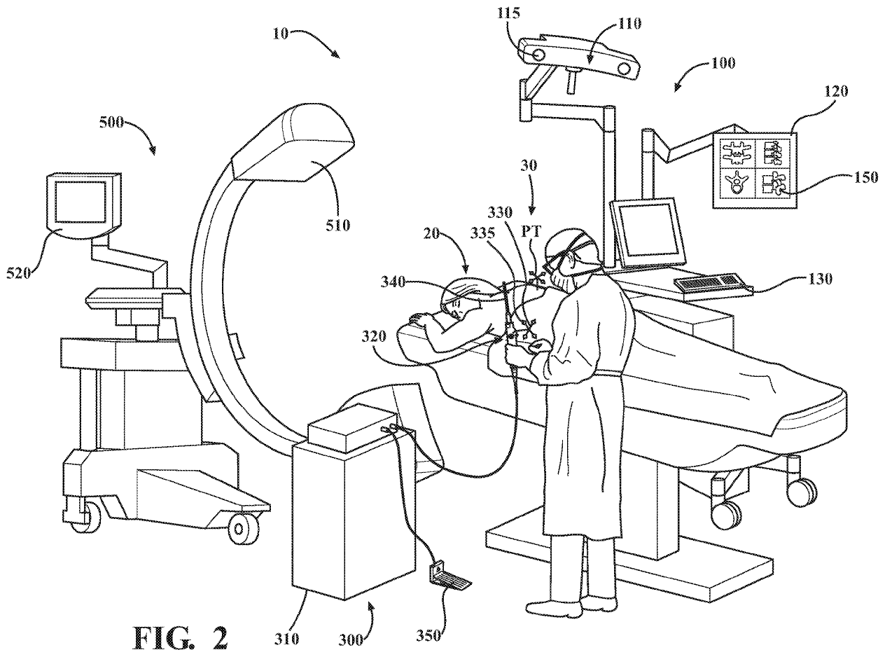

The surgical navigation system 100 may further include a navigation processor 140. The navigation processor 140 can be located on a personal computer or laptop computer. The navigation processor 140 may be in communication with the user input device(s) 130, the display unit(s) 120, a central processing unit (CPU) and/or other processors (e.g., instrument processors 215, 315, 415 of the respective instrument assemblies 200, 300, 400), memory (not shown), and storage (not shown). The navigation processor 140 may further include software and/or operating instructions related to the operation of the surgical navigation system 100 and configured upon execution to implement the various routines and/or methods disclosed herein. For instance, the software and/or operating instructions may include a planning system configured to define an accurate position and/or angular alignment of an implant in relation to the patient 20 (FIG. 2). The navigation processor 140 may be in wired or wireless communication with the surgical instrument assemblies 200, 300, 400, directly or indirectly.

The surgical navigation system 100 may further include software employed by the navigation processor 140 to control operation of the surgical assemblies 200, 300, 400, or more particularly the surgical instruments 220, 320, 420. The software may include a boundary generator. The boundary generator may be implemented on the navigation processor 140, one or more of the instrument processors 215, 315, 415, and/or on other components, such as on a separate processor or controller. An exemplary system for and method of boundary generation may be found in U.S. Patent Publ. No. 2004/0034283A1, which is hereby incorporated by reference herein in its entirety. The boundary generator may also be part of a separate system that operates remotely from the surgical system 10. The boundary generator is a software program or module that generates one or more virtual boundaries and/or one or more alert zones for constraining movement and/or operation of the surgical instruments 220, 320, 420. The virtual boundaries and/or zones may be associated with one or more regions of interest to a medical procedure, including, without limitation, anatomical features or regions targeted for manipulation, resection, or to receive a surgical implant, and/or anatomical features or regions to be avoided. In some examples, the boundary generator provides virtual boundaries that define a virtual drill and/or driver guide (e.g., a virtual implant planning guide). The virtual boundaries or alert zones may also be provided to control operation of the surgical instruments 220, 320, 420 relative to critical anatomical features that the surgeon wishes to avoid or manipulate, target depths, target areas to be resected, and/or target positions. The virtual boundaries may be one-dimensional (1D), two-dimensional (2D), three-dimensional (3D), and may include a point, line, axis, trajectory, plane (an infinite plane or plane segment bounded by the anatomy or other boundary), volume or other shapes, including complex geometric shapes. The virtual boundaries may be represented by pixels, point clouds, voxels, triangulated meshes, other 2D or 3D models, combinations thereof, and the like. U.S. Patent Publication No. 2018/0333207 and U.S. Pat. No. 8,898,043 are incorporated herein by reference in their entirety, and any of their features may be used to facilitate planning or execution of the surgical procedure. A plurality of boundaries may be used to define the zones likewise associated with the one or more regions of interest.

The virtual boundaries may be used in various ways. For example, the navigation processor 140 may control certain operations/functions of the surgical instruments 220, 320, 420 based on a relationship of the surgical instruments 220, 320, 420 to the boundaries and/or zones (e.g., spatial, velocity, etc.). Other uses of the boundaries and/or zones are also contemplated.

Boundaries to ensure that instruments are positioned at a desired depth may be defined by a virtual planar boundary, a virtual volumetric boundary, or other forms of virtual boundary. Virtual boundaries may also be referred to as virtual objects. The virtual boundaries may be defined with respect to an anatomical model, such as a 3D bone model. In other words, the points, lines, axes, trajectories, planes, volumes, and the like that are associated with the virtual boundaries may be defined in a coordinate system that is fixed relative to a coordinate system of the anatomical model such that tracking of the anatomical model (e.g., via tracking the associated anatomy to which it is registered) also enables tracking of the virtual boundary.

The anatomical model may be registered to a patient tracker PT such that the virtual boundaries become associated with the anatomical model and associated coordinate system. The virtual boundaries may be implant-specific, e.g., defined based on a size, shape, volume, etc. of an implant and/or patient-specific, e.g., defined based on the patient's anatomy. The virtual boundaries may be boundaries that are created pre-operatively, intra-operatively, or combinations thereof. In other words, the virtual boundaries may be defined before the surgical procedure begins, during the surgical procedure (including during tissue removal), or combinations thereof. The virtual boundaries may be provided in numerous ways, such as by the navigation processor 140 creating them, receiving them from other sources/systems, or the like. The virtual boundaries may be stored in memory for retrieval and/or updating.

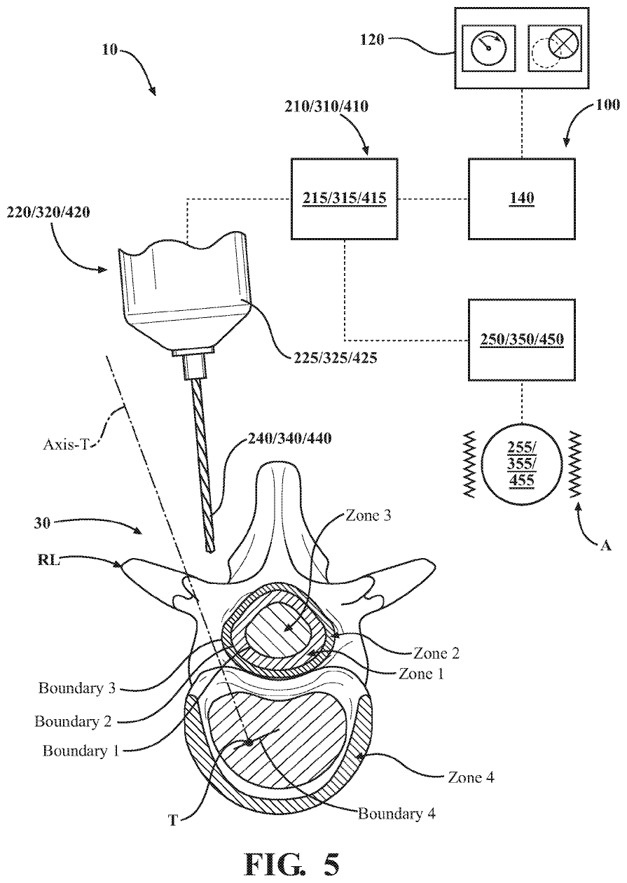

It is further contemplated that in some cases, the virtual boundaries may include multiple planar boundaries that can be used to delineate multiple target depths (e.g., three target depths) for separate instruments to be used in a single procedure. For example, the multiple virtual boundaries may include a first virtual boundary representing a target depth for a drill to bore a hole in a region of interest, a second virtual boundary representing a target depth for a tap relative to the region of interest, and a third virtual boundary representing a target depth for a driver to insert a screw relative to the region of interest, as are illustrated in FIG. 5 and explained in greater detail below. These multiple virtual boundaries can be activated, one at a time, by the navigation processor 140 to constrain cutting to one plane at a time. The navigation processor 140 can track the state of the surgical instruments 220, 320, 420 relative to the virtual boundaries.

The surgical navigation system 100 may also include a tracking unit 110. The tracking unit 110 may be configured to cooperate with tracking devices 230, 330, 430 of the surgical instrument assemblies 200, 300, 400 and/or a patient tracker PT (FIG. 2) to generate tracking data indicative of a pose of surgical instruments 220, 320, 420 of the surgical instrument assemblies 200, 300, 400 and/or of the patient, or more particularly one or more regions of interest of the patient, in a known coordinate system, such a coordinate system specific to the tracking unit 110. To this end, the tracking unit 110 may include one or more sensors 115. The sensor(s) 115 may include cameras, such as CCD cameras, CMOS cameras, and/or optical image cameras, magnetic sensors, radio frequency sensors, or any other sensor adapted to detect and/or sense the positions and/or orientations (poses) of the tracking devices 230, 330, 430 of the surgical instrument assemblies 200, 300, 400 in the known coordinate system. The navigation processor 140 may then be configured to apply a transformation function to such positions and/or orientations based on a known relationship between the tracking devices 230, 330, 430 and the surgical instruments 220, 320, 420 in the known coordinate system to determine the positions and/or orientations of the surgical instruments 220, 320, 420 in the known coordinate system. Tracking of the positions and/or orientations of regions of interest of a patient may be similarly performed using one or more patient trackers PT disposed relative to the regions. Description of a suitable tracking unit 110, and the various localizers that it can utilize, may be found in U.S. Patent Publication No. 2017/0333137, which is hereby incorporated by reference herein in its entirety.

Referring to FIG. 1, various exemplary surgical instrument assemblies 200, 300, 400 are illustrated in communication with the surgical navigation system 100. Each of the various exemplary surgical instrument assemblies will be described in greater detail below. The surgical instruments assemblies 200, 300, 400 may be configured to be in wired and/or wireless communication with the surgical navigation system 100. Furthermore, each of the surgical instrument assemblies 200, 300, 400 may have a number of similar components capable of performing similar functions and/or operations. Similar components between each of the various surgical instrument assemblies 200, 300, 400 will include the same two-digit number with a leading 2, 3, or 4 to reflect the associated surgical instrument assembly 200, 300, 400. For example, each of the surgical instrument assemblies 200, 300, 400 may include a surgical instrument 220, 320, 420.

The surgical system 10 may include a first surgical instrument assembly 200 in communication with the navigation system 100. For example, the first surgical instrument assembly 200 may be realized as a first surgical instrument 220, such as a surgical drill or driver, including a handpiece 225. The handpiece 225 may include a housing 210 configured to house the components of the first surgical instrument 220. The handpiece 225 may be shaped to define a handle or grip portion for the surgeon to hold while performing a medical procedure. Suitable handpieces are described in U.S. Pat. No. 5,747,953, which is hereby incorporated by reference herein in its entirety.

The first surgical instrument 220 may further include a first instrument processor 215 and a drive motor 245. Each of the first instrument processor 215 and the drive motor 245 may be disposed within the handpiece 225 of the first surgical instrument 220. The first instrument processor 215 and the drive motor 245 may be in communication with one another, and the first instrument processor 215 may be configured to control the operation of the drive motor 245, and by extension the first surgical instrument 220. For example, the first surgical instrument 220 may include an end-effector 240, such as a drill bit for boring a hole or a driver for inserting a screw into a region of interest. The end-effector 240 may be coupled to the handpiece 225 of the first surgical instrument 220 such that the drive motor 245 may be operably coupled to the end-effector 240. For example, the drive motor 245 may be configured to rotate a drill bit 240 to bore a hole and/or remove biological tissue from a region of interest. The first instrument processor 215 may be in communication with the drive motor 245 and configured to control operation of the drive motor 245, and by extension the drill bit 240. The first instrument processor 215 may also be in communication with the navigation processor 140 and configured to exchange data related to the position and/or orientation of the first surgical instrument 220, as well as data related to the operation of the first surgical instrument 220. For example, the first instrument processor 215 and the navigation processor 140 may be configured to communicate data between one another related to the operation of the first surgical instrument 220 based on the position and/or orientation of the first surgical instrument 220 as detected by the surgical navigation system 100.

The first surgical instrument assembly 200 may also include a power source 260. The power source 260 may be removably coupled to the handpiece 225 of the first surgical instrument 220. For example, the power source 260 may include a removable battery pack. It is also contemplated that the power source 260 may be formed as part of, or disposed within, the handpiece 225 of the first surgical instrument 220. The power source 260 may be in electrical communication with the first instrument processor 215 and/or the drive motor 245 and configured to selectively provide power to the drive motor 245 to rotate the end-effector 240. The power source 260 may also be a surgical console providing power to the first surgical instrument 220 with a cord.

In instances where the power source 260 takes the form of a removable battery pack, the power source 260 may further include a processor 265. The processor 265 may be in communication with the first instrument processor 215 via power signals and/or data signals. The processor 265 and the first instrument processor 215 may be configured to communicate between one another to control operation of the drive motor 245, and by extension the first surgical instrument 220. For example, the processor 265 in the power source 260 may be configured to identify when the power source 260 has dropped below a threshold charge level such that the power source 260 may be unable to continue operating the drive motor 245 at a minimum threshold for boring a hole or cutting biological tissue. The processor 265 may be configured to cut off all power to the first instrument processor 215 and/or the drive motor 245 to prevent operation of the end-effector 240 until the power source 260 has a sufficient charge level to operate the drive motor 245 at a rate above the minimum threshold for boring a hole or cutting biological tissue. The processor 265 in the power source 260 may also be in wireless communication with the navigation processor 140. To this end, the power source 260 may include a transceiver configured to send and receive signals between the power source 260 and the surgical navigation system 100 and/or the instrument processor 215.

The processor 265 and the navigation processor 140 may be configured to communicate data between one another related to the operation of the first surgical instrument 220 based on the position and/or orientation of the first surgical instrument 220 as detected by the surgical navigation system 100. For example, the navigation system 100 may be configured to communicate data to the processor 265 including instructions for the processor 265 to discontinue providing energy to the first instrument processor 215 and/or the drive motor 245 based on the position and/or orientation of the first surgical instrument 220 as detected by the surgical navigation system 100. The navigation system 100 may also be configured to communicate data to the processor 265 including instructions for the processor 265 to continue and/or resume providing energy to the first instrument processor 215 and/or the drive motor 245 based on the position and/or orientation of the first surgical instrument 220 as detected by the surgical navigation system 100.

The first surgical instrument assembly 200 may also include a switch 250, such as a trigger or button or lever, that is operably coupled to the first instrument processor 215. The switch 250 may be configured to be manipulatable by the medical professional to control energization of the variable speed drive motor 245. For example, the switch 250 may be manipulatable between a first position, a deenergized state, and a second position, an energized state. The first surgical instrument assembly 200 may also include a switch sensor that is configured to detect the position of the switch 250 and produce and/or communicate a signal indicative of the position of the switch 250 to the first instrument processor 215 based on a user's manipulation of the switch 250 to control the operation of the first surgical instrument 220. For example, the switch 250 may include a first position, a second position and a plurality of intermediary positions between the first and second positions. The first position may be configured as an off position, such that when the first instrument processor 215 receives a signal that the switch sensor has detected that the switch 250 is in the first position, the first instrument processor 215 prevents the flow of energy from the power source 260 to the drive motor 245, preventing the operation of the first surgical instrument 220. Conversely, the second position and the plurality of intermediate positions may each be configured as an on position. When the first instrument processor 215 receives a signal that the switch sensor has detected that the switch 250 is in the second position, the first instrument processor 215 may be configured to allow maximum flow of energy from the power source 260 to the drive motor 245, allowing the first surgical instrument 220 to operate at a maximum drilling or cutting speed. When the first instrument processor 215 receives a signal that the switch sensor has detected that the switch 250 is in one of the intermediary positions, the first instrument processor 215 may be configured to allow the flow of energy from the power source 260 to the drive motor 245 at a level corresponding to the position of the switch 250 between the first and second positions, allowing the first surgical instrument 220 to operate at an intermediate drilling or cutting speed. For example, if the first instrument processor 215 receives a signal that the switch sensor has detected that the switch 250 is positioned half-way (50%) between the first and second positions, the first instrument processor 215 may be configured to allow the flow of energy from the power source 260 to the drive motor 245 at a level that allows the first surgical instrument 220 to operate at a rate of 50% of the maximum drilling or driving speed. Alternatively, the first instrument processor 215 may be configured to allow the maximum flow of energy from the power source 260 to the drive motor 245 whenever the switch 250 is in a position other than the first position, allowing the first surgical instrument 220 to operate at the maximum drilling or cutting speed when the switch 250 is in the second position or any of the intermediary positions. An exemplary switch sensor may be found in U.S. Pat. No. 9,295,476, which is hereby incorporated by reference herein in its entirety.

The first surgical instrument assembly 200 may also include a first alert module 255. The first alert module 255 may be configured to indicate a position of the first surgical instrument 220 relative to a region of interest in a surgical procedure, such as a tissue region to be avoided, a tissue region to be manipulated, a tissue region to be resects, or a region to receive a surgical implant. More particularly, the first alert module 255 may be configured to indicate when the end effector 240 of the first surgical instrument 220, 320, 420 comes into a predetermined distance of or crosses a boundary associated with a region of interest. To this end, the first alert module 255 may include an audible, a tactile, and/or a visually perceptible device. The first alert module 255 may alternatively be a software routine. The first alert module 255 may be configured to be in communication with the first instrument processor 215 or the processor 265 of the power source 260. The first instrument processor 215, processor 265, or other processor of the surgical system 10 (e.g., navigation processor 140) may be configured to send a signal to activate the first alert module 255 to provide a warning or notification based on a pre-programmed condition or setting.

For example, as described above, the surgeon may use the user input device(s) 130 to enter defined conditions and/or settings into the surgical navigation system 100, such as selecting cortical walls, nerves, blood vessels, or similar anatomical structures that the surgeon wishes to avoid, and establish regions or zones (e.g., no-cut zones) surrounding those anatomical structures. The surgeon may also use the user input device(s) 130 to select and/or input a target location (position(s)), a target area for resection, a target trajectory in one or more degree of freedoms, or similar features of the surgical pathway to help guide the surgeon in performing the medical procedure. The first instrument processor 215, based on data provided by the navigation processor 140, may be configured to send a signal to activate the first alert module 255 upon the end-effector 240 of the first surgical instrument 220 entering one of the regions and/or zones, as defined by the surgeon. The first instrument processor 215 or other processor, based on data provided by the navigation processor 140, may also be configured to send a signal to activate the first alert module 255 upon the end-effector 240 of the first surgical instrument 220 being off trajectory and/or upon the end-effector 240 reaching the target location/area.

In an exemplary configuration, the first alert module 255 may include a vibrating device that is placed in contact with the surgeon and configured to vibrate to notify the surgeon of a particular condition or to provide a warning. In an exemplary configuration, as illustrated in FIG. 1, the first alert module 255 may include a vibrating device coupled to the switch 250 for controlling the operation of the first surgical instrument 220. The first alert module 255 may be configured to vibrate upon the occurrence of a defined condition. As the surgeon would be in constant contact with the switch 250 when actuating the first surgical instrument 220, the surgeon would feel the first alert module 255 vibrate and be notified of the occurrence of the defined condition. The first alert module 255 may be configured to produce a vibration in a specific pattern or interval upon the occurrence of the defined condition. Alternatively, the first alert module 255 may be configured to produce a first vibration in a specific pattern or interval upon the occurrence of a first condition and produce a second vibration in a different pattern or interval upon the occurrence of a second condition.

The first alert module 255 may also be configured as an audible device, such as a speaker, configured to provide an audible alert to the surgeon upon the occurrence of the defined condition. For example, the first alert module 255 may include a speaker configured to produce a specific sound upon the occurrence of the defined condition. Alternatively, the first alert module 255 may include a speaker configured to produce a sound in a specific pattern or interval upon the occurrence of the defined condition. The speaker may be included as part of the surgical navigation system 100.

In yet another configuration, the first alert module 255 may be configured as a visually perceivable device or indicator, such as a visual display, configured to provide a visual alert to the surgeon upon the occurrence of the defined condition. For example, the first alert module 255 may include a light configured to blink upon the occurrence of the defined condition. Alternatively, the first alert module 255 may include a plurality of multi-colored lights configured to light up and/or blink in a defined color or pattern upon the occurrence of the defined condition. In instances where the first alert module 255 is a display, the display may be configured to generate visual cues to indicate an alert condition. The navigation display unit 120 may be utilized as the display for the first alert module 255 such that the navigation display unit 120 is configured to provide a visual cue to alert the surgeon. For example, the navigation display unit 120 may be configured to display a prompt or window when the first alert module 255 is triggered providing a notification to the surgeon. Alternatively, the navigation display unit 120 may be configured to flash and/or change color when the first alert module 255 is triggered. One of the many advantages of utilizing the navigation display unit 120 as the display of the first alert module 255 is that the surgeon will already be regularly watching the navigation display unit 120 during execution of the procedure, so the surgeon is likely to receive the visual notification promptly if the navigation display unit 120 is configured to display the notification provided by the first alert module 255.

The removable power source 260 may also include the first alert module 255 in certain configurations. For example, the removable power source 260 may include a vibratory motor or a speaker responsive to signals generated by the navigation processor 140.

It is also contemplated that the first alert module 255 may include a combination of audible, tactile, and/or visually perceptible devices. For example, the first alert module 255 may be configured as a combination of an audible device and a tactile device, such that the tactile device may be configured to vibrate to provide a first alert and the audible device may be configured to produce a noise to provide a second alert. The first and second alerts may be indicative of an occurrence of the same defined conditions, or the first and second alerts may be indicative of an occurrence of distinct defined conditions. For example, the first alert may be based on the first surgical instrument 220 being misaligned with the target trajectory, and the second alert may be based on the end-effector 240 reaching the target location.

It is further contemplated that the first alert module 255 may be configured as a software routine configured to control the audible, tactile, and/or visually perceptible device. The software routine may also be configured to control a combination of the audible, tactile, and/or visually perceptible devices. For example, the software routine may include instructions stored in memory accessible by surgical navigation system 100, one or more of the surgical instrument assemblies 200, 300, 400, and/or other elements of the surgical system 10.

While the first alert module 255 is illustrated as being coupled to or proximate the switch 250 of the first surgical instrument assembly 200, it is contemplated that the first alert module 255 may be coupled to and/or positioned in alternative positions. For example, when the first alert module 255 includes a tactile device, the first alert module 255 may be configured as a vibrating member that is removably attached to the surgeon. The first alert module 255 may be configured as a wearable device, such as a bracelet to be worn on the surgeon's wrist or arm so that the surgeon would be able to feel the first alert module 255 vibrating upon the occurrence of the defined condition. Alternatively, when the first alert module 255 includes an audible device, the first alert module 255 may be configured as a speaker that is removably attached to the surgeon. The first alert module 255 may be configured as a Bluetooth speaker or earpiece to be worn on the surgeon's head or positioned within the surgeon's ear so that the surgeon would be able to hear the first alert module 255 producing a noise upon the occurrence of the defined condition.

While not required, there are a number of advantages to positioning the first alert module 255 away from the first surgical instrument 220. For example, one advantage of positioning the first alert module 255 away from the first surgical instrument 220 is that it may reduce the size of the first surgical instrument 220. This may allow for the first surgical instrument 220 to fit into smaller spaces. A smaller first surgical instrument 220 may also provide a less obstructed view of the surgical site for the surgeon. Another advantage of positioning the first alert module 255 away from the first surgical instrument 220, particularly in the case of a tactile device, is that the first alert module 255 will not vibrate or impact the movement of the first surgical instrument 220 while still providing an alert or notification to the surgeon. During highly technical procedures, an alert that vibrates the first surgical instrument 220 could cause the surgeon to move the first surgical instrument 220 in an undesirable position as a result of being startled by the first alert module 255 and/or the vibration imparting an undesirable movement to the first surgical instrument 220.

The first surgical instrument assembly 200 may also include a tracking device 230. The tracking device 230 may be coupled to the handpiece 225 of the first surgical instrument 220. The tracking device 230 may include a plurality of markers 235 that are identifiable by the tracking unit 110 of the surgical navigation system 100. The markers 235 may include passive tracking elements (e.g., reflectors) for transmitting light signals (e.g., reflecting light emitted from the tracking unit 110) to the sensor(s) 115. In other configurations, the markers 235 may be configured as active tracking markers, such as LEDs. It is also contemplated that the markers 235 may include a combination of active and passive arrangements. The markers 235 may be arranged in a defined or known position and orientation relative to the other markers 235 in order to allow the surgical navigation system 100 to determine the position and orientation (pose) of the surgical instrument 220, such as relative to a defined region of interest. For example, the markers 235 may be registered to the first surgical instrument 220 to allow the surgical navigation system 100 to determine the position and/or orientation of an end-effector 240 or cutting portion of the first surgical instrument 220 within a defined space, such as the surgical field. In one exemplary configuration, the surgical navigation system 100 may be configured to determine the position and/or orientation of the end-effector 240 or cutting portion of the second surgical instrument 220 relative to the target trajectory and/or the target location of the planned surgical pathway. In another exemplary configuration, the surgical navigation system 100 may also be configured to determine the position and/or orientation of the end-effector 240 or cutting portion of the second surgical instrument 220 relative to critical anatomical structures within the patient's body, as well as relative to user-defined boundaries, zones, and/or regions.

The surgical system 10 may additionally or alternatively include a second surgical instrument assembly 300 to be used with the navigation system 100. For example, the second surgical instrument assembly 300 may include a second surgical instrument 320, such as a high-speed surgical bur, including a handpiece 325. The handpiece 325 may be coupled to a console 310 that is configured to control the operation of various components of the second surgical instrument 320. The handpiece 325 may be shaped to define a handle or grip portion for the surgeon to hold while performing a medical procedure. Exemplary second surgical instruments that connect to consoles may be found in U.S. Pat. No. 10,016,209 and U.S. Patent Publication No. 20190117322, which are each hereby incorporated by reference herein in their entirety.

The second surgical instrument 320 may further include a second instrument processor 315 and a drive motor 345. The second instrument processor 315 may be disposed within the console 310 of the second surgical instrument assembly 300. The drive motor 345 may be disposed within the handpiece 325 of the second surgical instrument 320. The second instrument processor 315 and the drive motor 345 may be in communication with one another and the second instrument processor 315 may be configured to control the operation of the drive motor 345, and by extension the second surgical instrument 320. For example, the second surgical instrument 320 may be coupled to the console by a cord connecting the second instrument processor 315 to the drive motor 345 to allow communication between the second instrument processor 315 to the drive motor 345 to control operation of the drive motor 345. The second instrument processor 315 may also include an end-effector 340, such as a high-speed cutting bur. The end-effector 340 may be coupled to the handpiece 325 of the second surgical instrument 320 such that the drive motor 345 may be operably coupled to the end-effector 340. For example, the drive motor 345 may be configured to actuate the high-speed cutting bur 340 to grind and/or remove biological tissue from the surgical site corresponding to a region of interest. The second instrument processor 315 may be in communication with the drive motor 345 and configured to control the operation of the drive motor 345, and by extension the high-speed cutting bur 340. The second instrument processor 315 may also be in communication with the navigation processor 140 and configured to exchange data related to the position and/or orientation of the second surgical instrument 320, as well as data related to the operation of the second surgical instrument 320. For example, the second instrument processor 315 and the navigation processor 140 may be configured to communicate data between one another related to the operation of the second surgical instrument 320 based on the position and/or orientation of the second surgical instrument 320 as detected by the surgical navigation system 100. It is also contemplated that additional surgical instruments may be coupled to the console and/or in communication with the second instrument processor 315 disposed within the console 310.

The second surgical instrument assembly 300 may also include a power source (not shown). The power source may be coupled to the console 310 of the second surgical instrument assembly 300 and configured to provide energy to the drive motor 345 of the second surgical instrument 320 to actuate the end-effector 340. It is also contemplated that the console 310 may include a cord configured to be plugged into an outlet that is connected to an electrical grid for supplying energy to the second surgical instrument assembly 300. The power source may be in electrical communication with the second instrument processor 315 and/or the drive motor 345 and configured to selectively provide power to the drive motor 345 to actuate the end-effector 340.

The second surgical instrument assembly 300 may also include a switch 350, such as a footswitch, trigger, or button which is operably coupled to the second instrument processor 315. The switch 350 may be configured to produce and/or communicate a signal to the second instrument processor 315 based on a user input to control the operation of the second surgical instrument 320. For example, the switch 350 may include a first position, a second position and a plurality of intermediary positions between the first and second positions. The first position may be configured as an off position, such that when the first instrument processor 315 detects that the switch 350 is in the first position, the first instrument processor 315 prevents the flow of energy from the power source to the drive motor 345, preventing the operation of the second surgical instrument 320. Conversely, the second position and the plurality of intermediate positions may each be configured as an on position. When the first instrument processor 315 detects that the switch 350 is in the second position, the second instrument processor 315 may be configured to allow maximum flow of energy from the power source to the drive motor 345, allowing the second surgical instrument 320 to operate at a maximum speed, such as a maximum cutting speed or grinding speed. When the second instrument processor 315 detects that the switch 350 is in one of the intermediary positions, the second instrument processor 315 may be configured to allow the flow of energy from the power source to the drive motor 345 at a level corresponding to the position of the switch 350 between the first and second positions, allowing the second surgical instrument 320 to operate at an intermediate cutting or grinding speed. For example, if the second instrument processor 315 detects that the switch 350 is positioned half-way (50%) between the first and second positions, the second instrument processor 315 may be configured to allow the flow of energy from the power source to the drive motor 345 at a level that allows the second surgical instrument 320 to operate at a rate of 50% of the maximum cutting or grinding speed. Alternatively, the second instrument processor 315 may be configured to allow the maximum flow of energy from the power source to the drive motor 345 whenever the switch 350 is in a position other than the first position, allowing the second surgical instrument 320 to operate at the maximum cutting or grinding speed when the switch 350 is in the second position or any of the intermediary positions.

While not illustrated in the figures, it is contemplated that a plurality of surgical instruments 320 may be coupled to the console 310 and controlled by a footswitch. The switch 350, such as a footswitch, may be configured to control each of the plurality of surgical instruments. For example, a single footswitch may include a plurality of buttons, each of which may assigned to one of the plurality of surgical instruments. An exemplary surgical system including a switch connected to a console for controlling a plurality of surgical instruments is disclosed in U.S. patent application Ser. No. 15/450,477, which is hereby incorporated by reference herein in its entirety.

The second surgical instrument assembly 300 may also include a second alert module 355. The second alert module 355 may include an audible, a tactile, and/or a visually perceptible device. The second alert module 355 may be configured to be in communication with the second instrument processor 315 or directly with the navigation processor 140. The second instrument processor 315 or navigation processor 140 may be configured to send a signal to activate the second alert module 355 to provide a warning or notification based on a pre-programmed condition or setting.

For example, as described above, the surgeon may use the user input device(s) 130 to enter defined conditions and/or settings into the surgical navigation system 100, such as selecting cortical boundaries, nerves, blood vessels, or similar anatomical structures that the surgeon wishes to avoid and establish boundaries or zones (e.g., no-cut zones) surrounding those anatomical structures. The surgeon may also use the user input device(s) 130 to select and/or input a target location, a target area for resection, a target trajectory, or similar features to help guide the surgeon in performing the medical procedure. The second instrument processor 315, based on data provided by the navigation processor 140, may be configured to send a signal to activate the second alert module 355 upon the end-effector 340 of the second surgical instrument 320 entering one of the regions and/or zones, as defined by the surgeon, that surround an anatomical structure. For example, the surgeon may utilize the user input device(s) 130 of the surgical navigation system 100 to define a boundary or zone relative to the anatomical model. This may include identifying a critical anatomical feature such as a particular wall of the vertebral body, the central foramen, a nerve or blood vessel and assigning it a zone. The navigation system 100, as described above, may include a boundary generator for generating virtual boundaries within the patient relative to critical anatomical feature. As part of generating those boundaries, the navigation system 100 may be configured to recognize and/or define the virtual boundaries based on the segmentation algorithm. Upon the navigation system 100 generating one or more virtual boundaries, the navigation system 100 may be further configured to allow the surgeon to select a depth or distance. Upon the surgeon selecting a depth, the navigation system 100 may be configured to project a second virtual boundary at the selected depth or distance from the original virtual boundary. The area and/or volume defined between the original virtual boundary and the second virtual boundary may define at least portion of the zone. An exemplary system and/or method of segmentation may be found in U.S. Patent Publ. No. 2017/0061242A1, which is hereby incorporated by reference in its entirety.

The boundary generator may also identify additional zones that include regions or areas surrounding the critical anatomical feature, such as defining a second zone encircling the critical anatomical feature that is spaced a distance from the boundary of the critical anatomical feature. The boundary generator may also define additional subsequent zones, such as a third zone encircling the second zone and is spaced a distance from the boundary of the critical anatomical feature that is greater than the distance the second zone is spaced from the critical anatomical feature. In this exemplary configuration, the end-effector 340 is likely to contact the outermost alert zone first, triggering the second alert module 355 to produce a first alert. The end-effector 340 may then contact the next alert zone closest to the critical anatomical structure, triggering the alert module 355 to produce a second alert. The first and second alerts are configured to notify the surgeon of the occurrence of the end-effector 340 entering the respective alert zones assigned to the first and second alerts. The surgical navigation system 100 may be configured to allow the surgeon to define the alert zone(s) or region(s) as needed for the specific procedure. The alert zones may be configured as boundary lines or as regions encircling the critical anatomical structure. For example, the alert zone(s) may include a region or layer encircling the critical anatomical structure. The surgeon may define the thickness of the alert zone in the surgical navigation system 100. For example, a second alert zone located adjacent the critical anatomical structure may be defined as a two-millimeter-thick region surrounding the critical anatomical structure. The thickness may be varied based on the type of procedure and/or the surgeon's preference to ensure that the critical anatomical structure is not contacted. The surgeon may define a subsequent alert zone adjacent the second alert zone and opposite the critical anatomical structure, such that the subsequent alert zone is farther from the critical anatomical structure than the second alert zone.

The surgeon may define the subsequent alert zone as a five-millimeter-thick region encircling the outermost perimeter of the second alert zone. The thickness can be controlled through manipulation of a user input device based on the type of procedure and/or the surgeon's preference.

It should be appreciated that these alert zones may be automatically generated based on segmentation data from the patient scan.

The second instrument processor 315, based on data provided by the navigation processor 140, may also be configured to send a signal to activate the second alert module 355 upon the end-effector 340 of the second surgical instrument 320 being off trajectory and/or upon the end-effector 340 reaching the target location/zone/boundary. For example, the second alert module 355 may be activated to produce at least one of an audible, a tactile, or a visually perceptible alert based on the surgical navigation system 100 identifying that the end-effector 340 and/or the second surgical instrument 320 are not properly aligned with the target trajectory established as part of the panned surgical pathway. In this exemplary configuration, the second alert module 355 may produce a tactile alert, such as vibrating the switch 350 or removable power source, to notify the surgeon that the end-effector 340 is not properly aligned with the target trajectory. Once the end-effector 340 is properly aligned with the target trajectory, the second alert module 355 may be deactivated. The second alert module 355 may be similarly configured to be activated to produce at least one of an audible, a tactile, or a visually perceptible alert based on the surgical navigation system 100 identifying that the end-effector 340 and/or the second surgical instrument 320 have reached a target location as defined by the surgeon. For example, the second alert module 355 may produce a tactile alert, such as vibrating the switch 350, to notify the surgeon that the end-effector 340 has reached the target location/zone, such as a preferred depth or position relative to a critical anatomical boundary. Once the end-effector 340 has reached the target location, it is also contemplated that the control console may be configured to deactivate the drive motor 345, and by extension the end-effector 340, to prevent the end-effector 340 from going beyond the target location/zone.

In an exemplary configuration, the second alert module 355 may include a vibrating device that is placed in contact with the surgeon and configured to vibrate to notify the surgeon of a particular condition or to provide a warning. In an exemplary configuration, as illustrated in FIG. 1, the second alert module 355 may include a vibrating device coupled to the switch 350, such as a footswitch, for controlling the operation of the second surgical instrument 320. The second alert module 355 may be configured to vibrate upon the occurrence of a defined condition. For example, the second alert module 255 may include a vibrating device coupled to and/or in communication with the footswitch 350. In this configuration, the second alert module 355 may be configured to vibrate the footswitch 350 to notify the surgeon of the occurrence of the defined condition, such as the end-effector 340 of the second surgical instrument 320 approaching and/or entering one of the defined alert zones. As the surgeon would be in constant contact with the switch 350 when actuating the second surgical instrument 320, the surgeon would feel the second alert module 355 vibrate and be notified of the occurrence of the defined condition without affecting his or her grip on the handheld surgical instrument. The second alert module 355 may be configured to produce a vibration in a specific pattern or interval upon the occurrence of the defined condition. Alternatively, the second alert module 355 may be configured to produce a first vibration in a specific pattern or interval upon the occurrence of a first condition and produce a second vibration in a different pattern or interval upon the occurrence of a second condition. For example, the second alert module 355 may be configured to alternatingly vibrate and stop when the end-effector 340 of the second surgical instrument 320 approaches and/or enters a first alert zone, and the second alert module 355 may be configured to vibrate continuously when the end-effector 340 of the second surgical instrument 320 approaches and/or enters a second alert zone.

The second alert module 355 may also be configured as an audible device, such as a speaker, configured to provide an audible alert to the surgeon upon the occurrence of the defined condition. For example, the second alert module 355 may include a speaker configured to produce a specific sound upon the occurrence of the defined condition. Alternatively, the second alert module 355 may include a speaker configured to produce a sound in a specific pattern or interval upon the occurrence of the defined condition, such as the position of the end-effector crossing a defined zone/boundary.

In yet another configuration, the second alert module 355 may be configured as a visually perceivable device, such as a visual display, configured to provide a visual alert to the surgeon upon the occurrence of the defined condition. For example, the second alert module 355 may include a light configured to blink upon the occurrence of the defined condition. Alternatively, the second alert module 355 may include a plurality of multi-colored lights configured to light up and/or blink in a defined color or pattern upon the occurrence of the defined condition. The display may be integrated into the handpiece 325 or the battery or as part of the navigation system 100, or combinations thereof.

It is also contemplated that the second alert module 355 may include a combination of audible, tactile, and/or visually perceptible devices. For example, the second alert module 355 may be configured as a combination of an audible device and tactile device, such that the tactile device may be configured to vibrate to provide a first alert and the audible device may be configured to produce a noise to provide a second alert. The first and second alerts may be indicative of an occurrence of the same defined conditions, or the first and second alerts may be indicative of an occurrence of distinct defined conditions. For example, the first alert may be based on the second surgical instrument 320 entering a first region, and the second alert may be based on the end-effector 340 entering a second region.

It is further contemplated that the second alert module 355 may be configured as a software routine configured to control the audible, tactile, and/or visually perceptible device. The software routine may also be configured to control a combination of the audible, tactile, and/or visually perceptible devices. For example, the software routine may include instructions stored in memory accessible by surgical navigation system 100, one or more of the surgical instrument assemblies 200, 300, 400, and/or other elements of the surgical system 10.

While the second alert module 355 is illustrated as being coupled to the switch 350 of the second surgical instrument assembly 300, it is contemplated that the second alert module 355 may be coupled to and/or positioned in alternative positions. For example, when the second alert module 355 includes a tactile device, the second alert module 355 may be configured as a vibrating member that is removably attached to the surgeon. The second alert module 355 may be configured as a bracelet to be worn on the surgeon's wrist or arm so that the surgeon would be able to feel the second alert module 355 vibrating upon the occurrence of the defined condition. Alternatively, when the second alert module 355 includes an audible device, the second alert module 355 may be configured as a speaker that is removably attached to the surgeon. The second alert module 355 may be configured as a Bluetooth speaker or earpiece to be worn on the surgeon's head or positioned within the surgeon's ear so that the surgeon would be able to hear the second alert module 355 producing a noise upon the occurrence of the defined condition.

The second surgical instrument assembly 300 may also include a tracking device 330. The tracking device 330 may be coupled to the handpiece 325 of the second surgical instrument 320. The tracking device 330 be similar to as described above for the first surgical instrument assembly 200.

The surgical system 10 may include a third surgical instrument assembly 400 in communication with the navigation system 100. For example, the third surgical instrument assembly 400 may include a third surgical instrument 420, such as an ultrasonic instrument, including a handpiece 425. The handpiece 425 may be coupled to a console 410 that is configured to control the operation of various components of the third surgical instrument 420. The handpiece 425 may be shaped to include a handle or grip portion for the surgeon to hold while performing a medical procedure.

The third surgical instrument 420 may further include a third instrument processor 415 and a drive motor 445. The third instrument processor 415 may be disposed within the console 410 of the third surgical instrument assembly 400. The drive motor 445 may be disposed within the handpiece 425 of the third surgical instrument 420. The third instrument processor 415 and the drive motor 445 may be in communication with one another. The drive motor 445 may include a piezoelectric element configured to expand and contract upon the application of an electric current to the piezoelectric element. The piezoelectric element may include a plurality of disc-shaped piezoelectric elements arranged end to end in a stack. The third instrument processor 415 may be configured to control the operation of the drive motor 445, and by extension the third surgical instrument 420. For example, the third surgical instrument 420 may include an end-effector 440, such as an ultrasonic tip assembly. The end-effector 440 may include an ultrasonic tip assembly including a horn of which an ultrasonic tip portion vibrates at an ultrasonic wave velocity as the piezoelectric element(s) expand and contract. The ultrasonic tip assembly may also include an external sheath at least partially disposed over the horn except for the ultrasonic tip portion. The end-effector 440 may be coupled to the handpiece 425 of the third surgical instrument 420 such that the drive motor 445 may be operably coupled to the end-effector 440. For example, the drive motor 445 may be configured to actuate the ultrasonic tip assembly 440 to grind and/or remove biological tissue from the surgical site. The third instrument processor 415 may be in communication with the drive motor 445 and configured to control the flow of electric current to the piezoelectric element(s), controlling operation of the drive motor 445, and by extension the ultrasonic tip assembly 440. The third instrument processor 415 may also be in communication with the navigation processor 140 and configured to exchange data related to the position and/or orientation of the third surgical instrument 420, as well as data related to the operation of the third surgical instrument 420. For example, the third instrument processor 415 and the navigation processor 140 may be configured to communicate data between one another related to the operation of the third surgical instrument 420 based on the position and/or orientation of the third surgical instrument 420 as detected by the surgical navigation system 100.

The third surgical instrument assembly 400 may also include a power source (not shown). The power source may be coupled to the console 410 of the third surgical instrument assembly 400 and configured to provide energy to the drive motor 445 of the third surgical instrument 420 to actuate the end-effector 440. For example, the power source may include a removable battery pack. It is also contemplated that the console 410 may include a cord configured to be plugged into an outlet that is connected to an electrical grid for supplying energy to the third surgical instrument assembly 400. The power source may be in electrical communication with the third instrument processor 415 and/or the drive motor 445 and configured to selectively provide power to the drive motor 445 to actuate the end-effector 440.

The third surgical instrument assembly 400 may also include a switch 450, such as a footswitch, pedal or button, that is operably coupled to the third instrument processor 415, which may be similar to the switch 350 described above.

The third surgical instrument assembly 400 may also include a third alert module 455. The third alert module 455 may include an audible, a tactile, a visually perceptible device, and/or a software routine. The third alert module 455 may be configured to be in communication with the third instrument processor 415. The third instrument processor 415 may be configured to send a signal to activate the third alert module 455 to provide a warning or notification based on a pre-programmed condition or setting.

As described above, the surgeon may use the user input device(s) 130 to select and/or input a target location, a target area for resection, a target trajectory, or similar features of the surgical pathway to help guide the surgeon in performing the medical procedure, which may be used to establish target regions or zones. The third instrument processor 415, based on data provided by the navigation processor 140, may be configured to send a signal to activate the third alert module 455 upon the end-effector 440 of the third surgical instrument 420 entering one of the regions and/or zones, as defined by the surgeon, that surrounds an anatomical structure. The third instrument processor 415, based on data provided by the navigation processor 140, may also be configured to send a signal to activate the third alert module 455 upon the end-effector 440 of the third surgical instrument 420 being off trajectory and/or upon the end-effector 440 reaching the target location. For example, the third alert module 455 may be activated to produce at least one of an audible, a tactile, or a visually perceptible alert based on the surgical navigation system 100 identifying that the end-effector 440 and/or the third surgical instrument 420 are not properly aligned with the target trajectory established as part of the panned surgical pathway. In this exemplary configuration, the third alert module 455 may produce a tactile alert, such as vibrating the switch 450, to notify the surgeon that the end-effector 440 is not properly aligned with the target trajectory. Once the end-effector 440 is properly aligned with the target trajectory, the third alert module 455 may be deactivated. The third alert module 455 may be similarly configured to be activated to produce at least one of an audible, a tactile, or a visually perceptible alert based on the surgical navigation system 100 identifying that the end-effector 440 and/or the third surgical instrument 420 have reached a target location as defined by the surgeon in the panned surgical pathway. For example, the third alert module 455 may produce a tactile alert, such as vibrating the switch 450, to notify the surgeon that the end-effector 440 has reached the target location, such as preferred depth. Once the end-effector 440 has reached the target location, it is also contemplated that the console 410 may be configured to deactivate the drive motor 445, and by extension the end-effector 440, to prevent the end-effector 440 from going beyond the target location.

In an exemplary configuration, the third alert module 455 may be configured as described above for the first and second alert modules 255, 355.

The third surgical instrument assembly 400 may also include a tracking device 430. The tracking device 430 may be coupled to the handpiece 425 of the third surgical instrument 420. The tracking device 430 may be similar as defined above for the other instrument assemblies 200, 300 (e.g., including a plurality of markers 435, etc.).

The surgical instrument assemblies 200, 300, 400 described above are intended to be exemplary instruments and/or configurations within the surgical system 10 but are not intended to be limiting. Other types and forms of surgical instrument assemblies are contemplated. While a plurality of exemplary surgical instrument assemblies 200, 300, 400 are described as being a part of the surgical system 10 and in communication with the surgical navigation system 100, it is contemplated that the surgical system 10 may only include a single surgical instrument assembly 200, 300, 400 and a navigation system 100. Furthermore, while the surgical system 10 illustrated in FIG. 1 includes three surgical instrument assemblies 200, 300, 400 and a single surgical navigation system 100, it is contemplated that the surgical system 10 may be configured to include any combination of the surgical instrument assemblies 200, 300, 400, and/or the surgical navigation systems 100. For example, the surgical system 10 may include a single surgical instrument assembly 200, 300, 400 and a plurality of surgical navigation systems 100.

Referring to FIG. 2, an exemplary configuration of an operating room or surgical suite for performing a medical procedure on a patient 20 using the surgical system 10 described above is shown. The surgical system 10 including the surgical navigation system 100 and at least one of the surgical instrument assemblies 200, 300, 400 described above may be placed in the operating room surrounding the patient 20 and/or the surgical site 30 where the medical procedure is to be performed.

While only the second surgical instrument assembly 300 is illustrated in FIG. 2, it should be understood that it is only an exemplary configuration of the surgical system 10, and that it is contemplated that any number of surgical instrument assemblies 200, 300, 400 may be positioned within the operating room. As described above, the second surgical instrument assembly 300 includes the second surgical instrument 320 including the end-effector 340 and the tracking device 330. The tracking device 330 includes a plurality of markers 335 that are capable of being identified and/or tracked by the surgical navigation system 100. The second surgical instrument 320 is coupled to the console 310 that is positioned away from the second surgical instrument 320. The second surgical instrument assembly 300 also includes the switch 350 that is positioned away from the patient 20 and that is coupled to the console 310. The switch 350 is in communication with second surgical instrument 320 via the second instrument processor 315 (not shown) housed within the console 310.

While not previously discussed, it is also contemplated that the surgical system 10 may further include an imaging system 500, such as CT or MRI imaging device. The imaging system 500 may include a scanner 510 and a display unit 520. The scanner 510 may be utilized to take an image of the surgical site 30 on the patient 20 and display it on the display unit 520. For example, the scanner may include a C-arm configured to be rotated about the patient 20 to produce a plurality of images of the surgical site 30. The imaging system 500 may also include a processor (not shown) including software, as is known by those skilled in the art, which is capable of taking the plurality of images captured by the scanner 510 and producing a 2-D image and/or a 3-D model of the surgical site 30. The display unit 520 may be configured to display the resulting 2-D image and/or 3-D model.

The imaging system 500 may also be in communication with the navigation processor 140 of the surgical navigation system 100. The imaging system 500 may be configured to communicate via a wired and/or a wireless connection with the navigation processor 140. For example, the imaging system 500 may be configured to provide pre-operative and/or intra-operative image data, such as the resulting 2-D image and/or 3-D model of the surgical site 30, to the navigation processor 140. The navigation processor 140 may then be configured to provide the resulting 2-D image and/or 3-D model to the navigation display unit 120, where the surgeon, using the user input device(s) 130 or using algorithms, may identify and/or define the corresponding regions and/or zones around critical anatomical structures. For example, the surgeon may utilize the user input device(s) 130 of the surgical navigation system 100 to define an alert zone around a vertebral body, a nerve or a blood vessel that the surgeon wishes to avoid during execution of the medical procedure. The surgeon may utilize the user input device(s) 130 of the surgical navigation system 100 to input and/or modify the planned surgical pathway, boundaries, or alert zones to be utilized in executing the medical procedure.

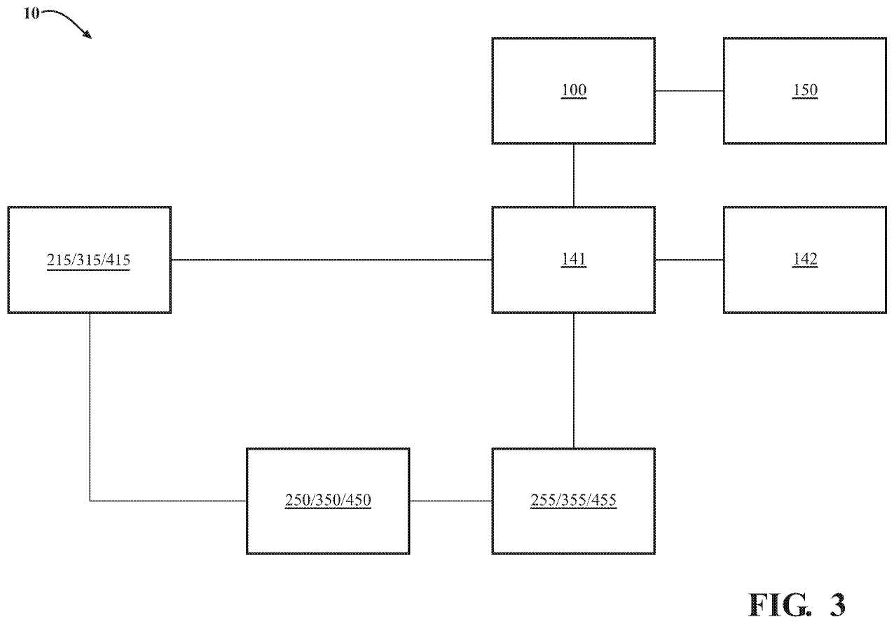

Referring to FIG. 3, an exemplary block diagram of the surgical system 10 is illustrated. According to the illustrated example, the surgical system 10 further includes a control system 141 in communication with the navigation system 100, or more particularly the navigation processor 140 of the navigation system 100, which in turn is in communication with the GUI 150. The control system 141 is also in communication with one or more of the instrument assemblies 200, 300, 400. Specifically, the control system 141 may be in communication with one or more of the surgical instruments 220, 320 420 and/or one or more of the consoles 310, 410 of the instrument assemblies 200, 300, 400, or more particularly one of the more the instrument processors 215, 315, 415 of these components. The control system 141 may further be in communication with one or more of the alert modules 255, 355, 455 of the instrument assemblies 200, 300, 400, which in turn may be in communication with one or more of the switches 250, 350, 450 of the instrument assemblies 200, 300, 400.

The control system 141 may include or be implemented by one or more controllers or one or more processors, each of which may be configured to operate under control of software routines or programs embodied by one or more computer-executable instructions stored in a memory accessible to the controller or processor. The computer-executable instructions may in turn be configured, upon execution by the one or more controllers or processors, to implement the functions, features, processes, and routines of the control system 141 described herein. As shown in the illustrated example, the control system 141 may be separate from and in communication with other components of the surgical system 10, such as the navigation system 100 and/or the instrument assemblies 200, 300, 400. In alternative implementations, the control system 141, or more particularly the functions, features, processes, and routines of the control system 141 described herein, may be implemented by one or more other components of the surgical system 10, such as the navigation system 100, or more particularly the navigation processor 140, and/or one or more of the instrument assemblies 200, 300, 400, or more particularly one or more of the instrument processors 215, 315, 415. In other words, the control system 141, or more particularly the functions, features, processes, and routines of the control system 141 described herein, may be distributed across multiple devices or systems of the surgical system 10, which may thus be considered as forming the control system 141.

The control system 141 may be configured to control the output of the navigation system 100 and the alert modules 255, 355, 455 in response to a tracked pose of the surgical instrument 220, 320, 420 in the known coordinate system relative to the virtual boundaries and/or zones. Further, the control system 141 may be configured to identify occlusion events (introduced below) and control the output of the navigation system 100 in response to the occlusion events. The control system 141 may also be configured to control the alert modules 255, 355, 455 and/or the drive motors 245, 345, 445 of the instrument assemblies 200, 300, 400 based on the occlusion events and/or based on one or more characteristics of the occlusion events. For instance, the control system 141 may be configured to adjust the drive speed of the drive motors 245, 345, 445 from a current non-zero value, such as set by the surgeon, to another non-zero value, which may be slower than the current non-zero value and/or generally unable to manipulate tissue in the regions of interest. Additionally or alternatively, the control system 141 may be configured to disable and thereby stop the drive motors 245, 345, 445 based on the occlusion events and/or based on one or more characteristics of the occlusion events.

Following control of the alert modules 255, 355, 455 and/or the drive motors 245, 345, 445 based on an occlusion event, the control system 141 may be configured to continue monitoring for occlusion events and maintain the state of the alert modules 255, 355, 455 and/or drive motors 245, 345, 445 until it detects, via updated tracking data from the tracking unit 110, that the occlusion event is no longer present, or alternatively until it receives input from the surgeon to disable occlusion monitoring. The control system 141 may then be configured to cease operation of the alert modules 255, 355, 455 and/or resume operation the drive motors 245, 345, 445. In some implementations, the surgeon may also be able to temporarily disable occlusion monitoring, such as for a predefined duration, and thereby cease operation of the alert modules 255, 355, 455 and/or resume operation the drive motors 245, 345, 445, such as by transitioning the relevant switch 250, 350, 450 to an off position and thereafter back to an on position.

In some examples, the surgical system 10 may also include a loss-of-localization-awareness (LLA) indicator 142 in communication with the control system 141. In such an example, the control system 141 is configured to control the LLA indicator 142 based on the existence of occlusion events as further described below. The LLA indicator 142 may be a visually perceptible device, such as a visual display, a light-emitting diode (LED), or any other suitable substitute. The LLA indicator 142 may also be an auditory and/or tactile device, or even a software routine. The software routine may be a set of instructions stored in memory and configured to control the audible, tactile, and/or visually perceptible device. The software routine may also be configured to control a combination of the audible, tactile, and/or visually perceptible devices. For example, the software routine may include computer-executable instructions stored in memory accessible by the surgical navigation system 100, one or more of the surgical instrument assemblies 200, 300, 400, and/or other elements of the surgical system 10.

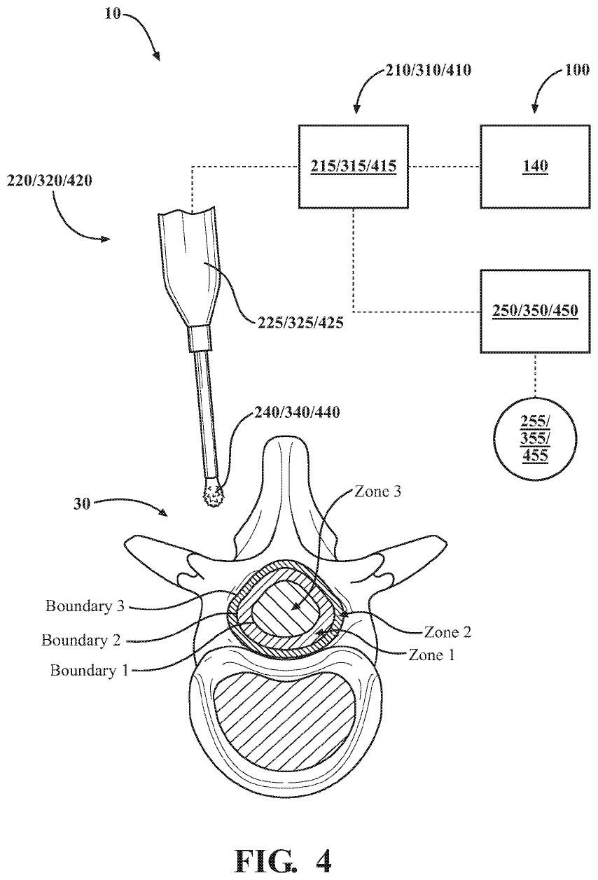

Referring to FIGS. 4 and 5, schematic views of the surgical system 10 described above during execution of a medical procedure are illustrated. The schematic views of the surgical system 10 include one of the surgical instruments 220, 320, 420 described above in various orientations relative to the patient 20, or more particularly a surgical site 30 of a patient 20, for the purpose of further explaining the operation of the surgical system 10.

As described above, the surgeon, using the surgical navigation system 100, may identify and/or define various boundaries, regions, target trajectories, target locations, or the like in the pre-operative and/or intra-operative patient data, such as a CT or MRI scan, that correspond to regions of interest to a medical procedure. For example, the surgeon may utilize the surgical navigation system 100 to select and/or define one or more virtual boundaries (Boundary 1, 2, 3, 4) and/or one or more alert zones (Zones 1, 2, 3, 4) relative to a critical anatomical structure or boundary, such as the central foramen, a wall of a vertebral body, a nerve or blood vessel, within the surgical site 30. This may include defining a plurality of virtual boundaries (Boundary 1, 2, 3, 4) and/or alert zones (Zones 1, 2, 3, 4) at varying distances from the critical anatomical structure and/or region of interest. For instance, in the example illustrated in FIGS. 4 and 5, the surgical site 30 includes a vertebra, on which a medical procedure is to be performed, and the virtual boundaries (Boundary 1, 2, 3, 4) may be defined relative to an outer perimeter of the spinal cord. The virtual boundaries (Boundary 1, 2, 3, 4) may be defined manually by the surgeon using the navigation system 100. However, the virtual boundaries (Boundary 1, 2, 3, 4) may also be selected by the surgeon from a populated list of virtual boundaries provided by the boundary generator software of the navigation system 100. The boundary generator may also be configured to define one or more alert zones (Zones 1, 2, 3, 4) relative to the critical anatomical structure and/or region of interest. Exemplary methods of configuring and/or defining virtual boundaries and alert zones are described in in International Patent Publ. No. 2021/062373, which is hereby incorporated by reference herein in its entirety.

During a medical procedure to remove biological tissue from a surgical site, the end-effector 240, 340, 440 of a given surgical instrument 220, 320, 420 may approach one of the various virtual boundaries (Boundary 1, 2, 3, 4), alert zones (Zones 1, 2, 3, 4), and/or a target depth (T). As described above, the surgical navigation system 100 may be configured to track the position and/or orientation (pose) of the surgical instrument 220, 320, 420 relative to the various virtual boundaries (Boundary 1, 2, 3, 4), alert zones (Zones 1, 2, 3, 4), and/or target depth (T), and communicate a signal or instruction to the instrument processor 215, 315, 415 associated with the given surgical instrument 220, 320, 420 to activate the alert module 255, 355, 455 associated with the given surgical instrument 220, 320, 420 to notify the surgeon when the end-effector 240, 340, 440 approaches and/or enters one of the various virtual boundaries (Boundary 1, 2, 3, 4), alert zones (Zones 1, 2, 3, 4), and/or target depth (T). The alert module 255, 355, 455 may be configured to provide specific alerts or a combination of alerts depending on the type of alert assigned to the respective boundary (Boundary 1, 2, 3, 4) and/or alert zone (Zones 1, 2, 3, 4). The surgical navigation system 100 may also be configured to manipulate/control the speed of the drive motor 245, 345, 445 associated with the given surgical instrument 220, 320, 420, and by extension the speed at which the end-effector 240, 340, 440 is operated based on the position and/orientation of the surgical instrument 220, 320, 420 relative to the one or more of the virtual boundaries (Boundary 1, 2, 3, 4), alert zones (Zones 1, 2, 3, 4), and/or target depth (T). For example, the surgical navigation system 100 may be configured to deactivate or decelerate the drive motor 245, 345, 445 from a maximum cutting speed to a minimum cutting speed based on the position of the surgical instrument 220, 320, 420 relative to the one or more virtual boundaries (Boundary 1, 2, 3, 4), alert zones (Zones 1, 2, 3, 4), and/or target depth (T).

Once the surgical navigation system 100 has transmitted a signal to the processor 215, 315, 415 of the surgical instrument 220, 320, 420 to deactivate or decelerate the variable speed drive motor 245, 345, 445 upon the end-effector 240, 340, 440 being adjacent and/or distal to one of the virtual boundaries and/or entering one of the various alert zones, the processor 215, 315, 415 may be configured to decelerate or deactivate the variable speed drive motor 245, 345, 445, such as by causing the drive motor 245, 345, 445 to rotate at 0 rpms in the latter case. While the end-effector 240, 340, 440 remains adjacent and/or distal to one of the virtual boundaries and/or within one of the various alert zones, after temporarily deactivating or decelerating the variable speed drive motor 245, 345, 445, the processor 215, 315, 415 may be configured to reactivate the variable speed drive motor 245, 345, 445, such as to a speed prior to the deceleration or deactivation. In some instances, the variable speed drive motor 245, 345, 445 may be reactivated by the processor 215, 315, 415 following a defined period of time, such as the passage of 1, 2, 3, or 4 seconds. Alternatively, the variable speed drive motor 245, 345, 445 may be reactivated by the processor 215, 315, 415 following the processor 215, 315, 415 receiving a signal that the switch 250, 350, 450 associated with the given surgical instrument 220, 320, 420 has been manipulated by the user, such as from an on position to an off position and then back to the on position.

More specifically, the control system 141 may be configured to cause the alert module 255, 355, 455 to alert the surgeon and/or control the drive motor 245, 345, 445 if the end-effector 240, 340, 440 crosses and/or approaches the defined virtual boundaries (Boundary 1, 2, 3, 4), the defined alert zones (Zones 1, 2, 3, 4), and/or the defined target depth (T). The nested nature of boundaries 1, 2, 3, and 4 and zones 1, 2, 3, and 4 (one being more distal than the other relative to a region of interest) may provide progressively more alerts to ensure that the surgeon is aware that the surgical instrument 220, 320, 420 is in close proximity to the critical anatomical structure and/or a target depth (T) associated with the region of interest. The alert module 255, 355, 455 may be configured to provide specific alerts or a combination of alerts depending on the type of alert assigned to the respective alert zone (Zones 1, 2, 3, 4) and/or virtual boundaries (Boundary 1, 2, 3, 4).

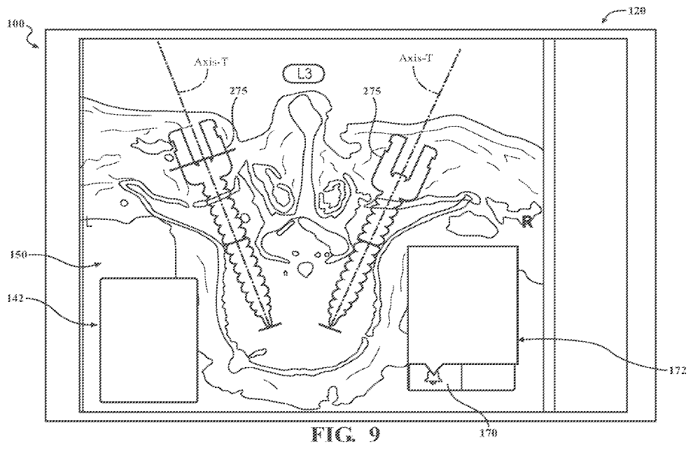

As will be appreciated from the figures (e.g., FIG. 5), the surgical instrument 220, 320, 420 may be configured to bore a hole to remove biological tissue from region of interest in the surgical site 30 and/or drive a screw, such as a pedicle screw, into the region of interest of surgical site 30. In this scenario, the surgeon may select and/or define a planned implant pose, such as one or more planned screw poses, including a target trajectory, Axis-T, and the target depth (T). Alternatively, the navigation processor 140 may receive a planned surgical pathway that was automatically generated based on the segmentation of the patient image data and the planned pose of the medical device or implant 275 (FIG. 9) to be inserted during the procedure. The control system 141 may be configured to cause the alert module 255, 355, 455 to alert the surgeon and/or control the drive motor 245, 345, 445 if the end-effector 240, 340, 440 deviates from the target trajectory Axis-T and/or crosses and/or approaches the target depth (T).

Exemplary methods of controlling instrument motors and/or alerting the surgeon based on instrument position relative to virtual boundaries, alert zones, target trajectories, and/or target depths is described in International Patent Publ. No. 2021/062373, which is incorporated by reference herein above.

During the course of tracking surgical instrument 220, 320, 420 and by extension the end-effector 240, 340, 440, it is contemplated that the tracking device 230, 330, 430 of the surgical instrument 220, 320, 420 and/or the patient tracker PT may be blocked from view of the navigation system 100 (e.g., the tracking unit 110). For example, where the surgical instrument 220, 320, 420 is a hand-held instrument, the surgeon may move the instrument 220, 320, 420 out of view of the tracking unit 110. In another example, the surgeon may step between the tracking unit 110 and the surgical instrument 220, 320, 420. In yet another example, the tracking device 230, 330, 430 associated with the given surgical instrument 220, 320, 420 may include active tracking markers powered by a power source 260 associated with the given surgical instrument 220, 320, 420. If the power supplied by the power source 260 is disrupted, the tracking device 230, 330, 430 may fail to output tracking signals to the tracking unit 110. In yet another example, the tracking unit 110 may fail to discern the pose of the surgical instrument 220, 320, 420 due to issues related to the tracking unit 110 itself. Regardless of the cause, the control system 141 may be configured to determine if the line-of-sight between the tracking unit 110 and any of the tracking devices 230, 330, 430 of the surgical instruments 220, 320, 420 and/or the patient tracker PT has been occluded, such as based on the tracking data received from the tracking unit 110 indicating detection of fewer than all of markers of the tracking devices 230, 330, 430. These occlusions are hereby referred to as occlusion events. The control system 141 may be generally in communication with the tracking unit 110, the alert modules 255, 355, 455, and the consoles 310, 410 (if applicable). If the surgical instrument assembly 200, 300, 400 does not include a console (e.g., surgical instrument 220), then the control system 141 may be in communication with the surgical instrument 220, 320, 420 and/or the instrument processor 215, 315, 415 associated with the surgical instrument 220, 320, 420.

The control system 141 may be configured to control the alert module 255, 355, 455 and/or the drive motor 245, 345, 445 associated with a given surgical instrument 220, 320, 420 subject to an occlusion event based on one or more characteristics of the occlusion event, which may include one or more characteristics of the occlusion event itself (e.g., the duration and/or cause of the occlusion event) and/or one or more characteristics of the surgical instrument 220, 320,420 associated with the occlusion event, such as during and/or prior to the occlusion event, or more particularly immediately prior to the occlusion event, which may correspond to one or more poses of the surgical instrument 220, 320, 420 last seen by the navigation system 100 prior to the occlusion event according to the tracked pose of the surgical instrument 220, 320, 420 in the known coordinate system. For example, the control system 141 may consider a cause of the occlusion event, a duration of the occlusion event, an orientation or pose of the surgical instrument 220, 320, 420 prior to the occlusion event, a distance between the instrument 220, 320, 420 and the virtual boundaries or regions of interest prior to the occlusion event, a direction of a movement of the instrument 220, 320, 420 relative to (e.g., towards or away from) a region of interest prior to the occlusion event, a speed, velocity, or acceleration of the instrument 220, 320, 420 prior to the occlusion event, a motion parameter of the instrument 220, 320, 420 prior to the occlusion event, and/or user input received during the occlusion event to control operation of the drive motor 245, 345, 445. Other factors not listed herein may be considered as well. As an example, and as expanded upon below, the control system 141 may determine to not alert the surgeon or change the operation of the instrument 220, 320, 420 if the occlusion event is shorter than a predetermined duration threshold while the instrument 220, 320, 420 is at least a certain distance away from the patient regions of interest and/or the virtual boundaries.

The characteristics of an occlusion event, such as characteristics of the occlusion event itself and/or of the surgical instrument 220, 320, 420 and associated with the occlusion event (e.g., during and/or prior to the occlusion event), are helpful indicators of whether the occlusion event has or may cause potentially unacceptable levels of uncertainty in the surgical system 10. As briefly noted above, the characteristics of an occlusion event may include: the cause of the occlusion event, the duration of the occlusion event, the pose of the surgical instrument 220, 320, 420 prior to the occlusion event, the distance between the instrument 220, 320, 420 and the virtual boundaries or regions of interest prior to the occlusion event, the orientation or direction of movement of the instrument 220, 320, 420 relative to a virtual boundary or region of interest prior to the occlusion event, the speed, velocity, or acceleration of the instrument 220, 320, 420 prior to the occlusion event, and/or a motion parameter of the instrument 220, 320, 420 prior to the occlusion event, among other characteristics. Depending on the specific circumstances present during usage of the surgical instrument 220, 320, 420, different characteristics may be considered by the control system 141.

In a first example, the control system 141 may be configured to control at least one of the drive motor 245, 345, 445 and the alert module 255, 355, 455 associated with the instrument 220, 320, 420 subject to an occlusion event based on the duration of the occlusion event alone. More specifically, the control system 141 may be configured to compare the duration of the occlusion event to a set duration threshold, and control at least one of the drive motor 245, 345, 445 and the alert module 255, 355, 455 based on the comparison. In this way, the control system 141 may be configured to ignore any occlusion events where the duration of the occlusion event is shorter than the duration threshold. If, on the other hand, the occlusion event lasts longer than the duration threshold, the control system 141 may alert the surgeon (e.g., with the alert module 255, 355, 455) and/or control the drive motor 245, 345, 445 of the surgical instrument 220, 320, 420. The duration threshold may be a specific amount of time, for example, 0.001 seconds. Alternatively, the duration threshold may be defined based on the type of surgical instrument 220, 320, 420 being used, and/or the procedure being performed with the instrument 220, 320, 420. For example, the duration threshold may be 0.001 seconds when operating on the patient's spine and 0.01 seconds when operating on the patient's knee.

In a second example, the control system 141 may be configured to control at least one of the drive motor 245, 345, 445 and the alert module 255, 355, 455 based on the tracked pose of the instrument 220, 320, 420 subject to the occlusion event and the virtual boundaries in the known coordinate system prior to the occlusion event, such as by determining a positional relationship or distance between the instrument and a region of interest based on the tracked pose of the instrument 220, 320, 420 and a virtual boundary associated with the region of interest. Specifically, the control system 141 may be configured to determine a distance between the surgical instrument 220, 320, 420 and each of the virtual boundaries in the known coordinate system, and identify a corresponding distance threshold for each distance. In such an example, responsive to the distance between the instrument 220, 320, 420 and each of the virtual boundaries associated with a region of interest being greater than the corresponding distance threshold, the surgical instrument 220, 320, 420 may be considered as less likely to have an unintended effect during an occlusion event, and the control system 141 may thus be configured to not alert of any occlusion events. Alternatively, responsive to the distance between the instrument 220, 320, 420 and one of the virtual boundaries associated with a region of interest being less than or equal to the corresponding distance threshold, the surgical instrument 220, 320, 420 may be considered as more likely to have an unintended effect during an occlusion event, and the control system 141 may thus be configured to alert the surgeon of any occlusion events via the alert module 255, 355, 455 and/or control the drive motor 245, 345, 445 associated with the occlusion event. The distance threshold may be a specific distance, for example, 1 millimeter. Alternatively, the distance threshold may be defined based on the type of surgical instrument 220, 320, 420 being used, and/or on the procedure being performed with the instrument 220, 320, 420, and/or on the region of interest associated with the virtual boundary. For example, the distance threshold may be 5-10 millimeters when operating on the patient's spine and 1 millimeter when operating on the patient's knee. The distance threshold may also be much larger (e.g., 1 meter) in cases where occlusion events are of increased concern to the surgeon and/or the patient.

The distance between the surgical instrument 220, 320, 420 and each of the virtual boundaries may be calculated as a line between the end effector 240, 340, 440 of the surgical instrument 220, 320, 420 and the closest point on the virtual boundary. In some cases, the line may be normal to a point on the mesh of the virtual boundary. Further, it should be appreciated that any of the distance calculations, including those described below, may be utilized to determine whether the surgical instrument 220, 320, 420 has crossed a virtual boundary and is now in an alert zone (e.g., Zones 1, 2, 3, or 4) and/or in a region of interest, and/or whether the surgical instrument 220, 320, 420 has come within the distance threshold relative to the virtual boundary such that the surgeon should be alerted of the occlusion event.

The distance between the surgical instrument 220, 320, 420 and the virtual boundaries may also be calculated via specific methods based on which type of end effector 240, 340, 440 is being driven by the instrument 220, 320, 420, or the type of instrument 220, 320, 420 being used. If the end effector 240, 340, 440 is a burr (e.g., FIG. 4), for example, the control system 141 may be configured to calculate the distance as the distance between the center of the head of the burr 240, 340, 440 and the virtual boundary, such as a point on the mesh of the virtual boundary. However, the distance may be a scalar/direction-less value such that the control system 141 is unsure whether the burr 240, 340, 440 is 1 mm away from or outside the virtual boundary or 1 mm into or inside the virtual boundary, which may correspond to being positioned within a region of interest. In order to determine whether the burr 240, 340, 440 is 1 mm away from or 1 mm into the virtual boundary, the control system 141 may utilize ray casting to determine a direction and thereby further distinguish the distance. The ray casting may include casting a ray from the center of the burr 240, 340, 440 and into the shaft of the burr 240, 340, 440 toward the handpiece 225, 325, 425 of the instrument 220, 320, 420, such as in the known coordinate system. If the ray collides with the virtual boundary, then the control system 141 may assume that the burr 240, 340, 440 is 1 mm into the virtual boundary and is thus in a potentially precarious position relative to the anatomy of the patient defining a region of interest. This is a safe assumption, because if the center of the burr 240, 340, 440 is inside of the volume bounded by the virtual boundary, the virtual boundary will surround at least a portion of the shaft of the burr 240, 340, 440. Thus, a ray cast into the shaft of the burr 240, 340, 440 will collide with the boundary if at least a portion of the shaft is inside of the boundary. Even if only the burr head 240, 340, 440 is inside of the boundary, a ray cast from the center of the burr will also collide with the boundary. On the other hand, if the ray does not collide with the virtual boundary, then the control system 141 may assume that the burr 240, 340, 440 is 1 mm away from the virtual boundary. In instances where the ray intersects the boundary, the control system 141 may be configured to control the alert module 255, 355, 455 to indicate as such. In such an example, the control system 141 may be configured allow the alert module 255, 355, 455 to continue alerting if the end effector 240, 340, 440 is within the boundary based on the intersection of the ray. In other words, the control system 141 may control the alert module 255, 355, 455 based on the intersection of the ray, the pose of the instrument 220, 320, 420, and the boundary.

Further, if the end effector 240, 340, 440 is a drill bit (e.g., FIG. 5), the control system 141 may perform a method involving ray casting similar to the above description. However, instead of calculating the distance from the center of a burr, the control system 141 may be configured to calculate the distance between the tip of the drill bit 240, 340, 440 and the virtual boundary. After which, the control system 141 may be configured to determine the direction of the distance by casting a ray from the tip of the drill bit 240, 340, 440 and into the remainder of the drill bit 240, 340, 440 toward the handpiece 225, 325, 425. Similarly, if the ray collides with the virtual boundary, the control system 141 may assume that the drill bit 240, 340, 440 is 1 mm into or inside the virtual boundary and is thus in a potentially precarious position relative to the anatomy of the patient defining a region of interest. Alternatively, if the ray does not collide with the virtual boundary, the control system 141 may assume that the drill bit 240, 340, 440 is 1 mm away from the virtual boundary. It should be understood that the specific distances used herein (e.g., 1 mm) are exemplary and the control system 141 may utilize the above method with respect to other distances. In instances where the ray intersects the boundary, the control system 141 may be configured to control the alert module 255, 355, 455 to indicate as such. In such an example, the control system 141 may allow the alert module 255, 355, 455 to continue alerting if the end effector 240, 340, 440 is within the boundary based on the intersection of the ray. In other words, the control system 141 may be configured to control the alert module 255, 355, 455 based on the intersection of the ray, the pose of the instrument 220, 320, 420, and the boundary.

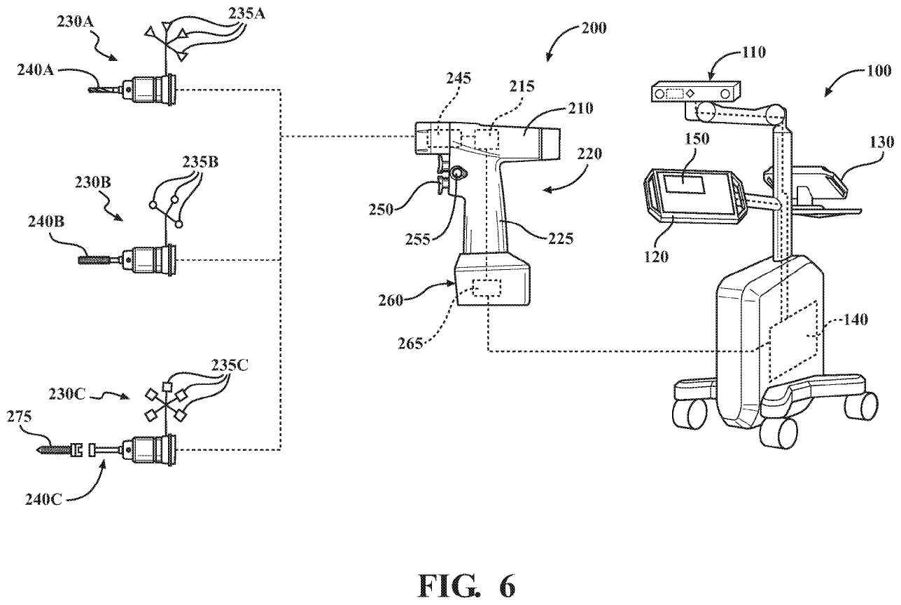

Even further, if the end effector 240, 340, 440 is a driver for driving a screw (e.g., FIG. 6), the control system 141 may be configured to calculate the distance as the distance between the tip of the driver 240, 340, 440 and the center of the planned position of the screw 275, which may likewise be represented by a virtual boundary. If the screw 275 is coupled to the driver 240, 340, 440 and being actively driven into the patient, the distance may instead be the distance between the tip of the screw 275 attached to the driver 240, 340, 440 and the center of the planned position of the screw 275. For example, FIG. 9 shows the planned position of the screw 275 relative to the patient's anatomy, which may define a region of interest. The center of the planned position of the screw 275 may be determined by bounding the planned position of the screw 275 with a spherical virtual boundary of diameter D equal to the length of the planned position of the screw 275 (e.g., as represented by a line segment spanning from the one end of the planned position of the screw 275 to the opposite end of the planned position of the screw 275), and then treating the center of the spherical virtual boundary as the center of the planned position of the screw 275. The control system 141 may be configured to determine for which planned screw 275 position to calculate the distance by bounding multiple planned screw 275 positions with spherical virtual boundaries and then determining distances between the driver 240, 340, 440 and each of the spherical virtual boundaries. The control system 141 may be configured to then calculate the distance based on whichever distance is the lowest (i.e., which planned screw 275 position the tip of the driver 240, 340, 440 is closest to). Alternatively, the control system 141 may be configured to calculate the distance as a plurality of distances, each of the plurality of distances corresponding to the distance between the tip of the driver 240, 340, 440 and the center of one of the spherical boundaries.

After the distance between the tip of the driver 240, 340, 440 and the center of the spherical virtual boundary is calculated, the radius R of the spherical virtual boundary may be subtracted from the distance. This way, the distance is adjusted to be the distance between the tip of the driver 240, 340, 440 and the outer bounds of the spherical virtual boundary, such as away from the region of interest. As an undesirable effect may be posed to the patient when the tip of the driver 240, 340, 440 is impinging on the virtual boundary, this adjusted distance may be desirable. Again, because the control system 141 may not determine a direction along with the distance, the spherical virtual boundary may be used to minimize the potential of such undesirable effect. If the driver 240, 340, 440 is approaching the planned screw 275 position from top of the planned screw 275 position, then the adjusted distance will coincide with the distance between the top of the planned screw 275 position and the tip of the driver 240, 340, 440. But if the driver 240, 340, 440 is approaching the planned screw 275 position from the side of the screw 275, then the adjusted distance will coincide with the distance between the driver 240, 340, 440 and center of the planned screw 275 position plus the radius R. In other words, the adjusted distance may be accurate when approaching the planned screw 275 position from the top, but otherwise inaccurate when approaching the planned screw 275 position from the side. Since the calculation may be directionless, the control system 141 may not know if the driver 240, 340, 440 is approaching from the top or the side of the planned screw 275. Therefore, it may be preferrable to assume that the driver 240, 340, 440 is approaching the top of the planned screw 275 position with the driver 240, 340, 440 coaxial with a longitudinal axis of the planned screw 275 position. Additionally, the distance calculation above may be applied to the plurality of distances where the control system 141 is configured to monitor the distance between the tip of the driver 240, 340, 440 and multiple planned screw 275 positions. Since a drill bit may be used prior to the driver 240, 340, 440, this method may also be utilized when a drill bit 240, 340, 440 is connected to the instrument 220, 320, 420 instead of the driver 240, 340, 440.

In the instance where there are multiple virtual boundaries for a particular patient, the control system 141 can perform distance calculations relative to all boundaries, or the control system 141 can perform the described calculations only for the boundary that is closest to the instrument 220, 320, 420. More specifically, the control system 141 can determine the closest boundary based on the boundary that is closest to the current pose of instrument 220, 320, 420. This determination can be based on the center of the planned screw 275 position and the closest point on the mesh of the boundary, or using the center of the burr 240, 340, 440 and the closest point on the mesh of the boundary. The control system 141 may be configured to switch between boundaries as the instrument 220, 320, 420 is moved by the user to another aspect of the procedure, such as based on when the user is placing a different screw 275 or when the user is resecting a different level vertebra. In other implementations, the control system 141 may be configured to perform distance calculations only for the boundary that is closest to an anatomical structure defining a region of interest.

In a third example, the control system 141 may be configured to control at least one of the drive motor 245, 345, 445 and the alert module 255, 355, 455 based on both the duration of the occlusion event and the distance between the instrument 220, 320, 420 and the virtual boundaries and/or regions of interest prior to the occlusion event. The control system 141 may be configured to determine the duration threshold along with the distance threshold and control the drive motor 245, 345, 445 and/or the alert module 255, 355, 455 based on one or more other characteristics of the occlusion event relative to a combination of the duration and distance thresholds. By considering both the duration and the distance associated with the occlusion event, the control system 141 can better predict whether the occlusion event has or is causing unacceptable levels of uncertainty. The control system 141 may thus be configured to ignore a longer occlusion event if the surgical instrument 220, 320, 420 is far away from the virtual boundaries and/or region of interest. For example, the control system 141 may ignore the occlusion event if the instrument 220, 320, 420 was last seen 2 meters from the virtual boundaries, unless the occlusion event lasts longer than 10 seconds.

In the above combinational example, the control system 141 may also be configured to set the duration threshold based on the distance between the instrument 220, 320, 420 and the regions of interest and/or virtual boundaries, or set the distance threshold based on the duration of an occlusion event. For instance, as the instrument 220, 320, 420 is moved closer to the regions of interest and/or virtual boundaries, the duration threshold may be lowered. Alternatively, the duration threshold may be increased as the instrument 220, 320 420 is moved farther away from the regions of interest and/or virtual boundaries. This allows the control system 141 to determine whether to alert the surgeon and/or control the drive motor 245, 345, 445 based on the potential risks associated with the occlusion event. For example, if the instrument 220, 320, 420 is relatively far from the regions of interest and/or virtual boundaries, it may not be as valuable for the surgeon to know that the instrument 220, 320, 420 is occluded from view of the tracking unit 110, even for a relatively significant amount of time, compared to when the instrument 220, 320, 420 is relatively near a region of interest and/or a virtual boundary. This is especially true where the instrument 220, 320, 420 is not currently being used. The surgeon may have set the instrument 220, 320, 420 down while performing other tasks, and he/she may not benefit from knowing that the occlusion event is occurring for the instrument 220, 320, 420 which is not currently in use.

For instance, the control system 141 may be configured to determine the distance between the instrument 220, 320, 420 and a region of interest or a virtual boundary associated with the region interest based on the tracked pose of the instrument in the known coordinate system relative to the virtual boundary in the known coordinate system, such as described above, and determine whether the distance between the instrument and the region of interest or virtual boundary is greater than a predefined threshold distance. If so, then the control system 141 may be configured to set the duration threshold to a first predefined duration, and if not, then the control system 141 may be configured to set the duration threshold to a second predefined duration that is less than the first predefined duration threshold. In this way, as the instrument 220, 320, 420 moves closer to a region of interest or associated virtual boundary, the control system 141 may be configured to reduce the minimum duration in which the occlusion event needs to be present to take action (e.g., control the drive motor 245, 345, 445 and/or the alert module 255, 355, 455).

In some examples, multiple virtual boundaries may be associated with a given region of interest, with a first of the virtual boundaries bounding the region of interest, and a second of the virtual boundaries being located farther form the region of interest than the first boundary, such as according to a predefined distance from the first boundary. In this case, the control system 141 may be configured to determine whether the end effector 240, 340, 440 of a surgical instrument 220, 320, 420 is between the first and second boundaries or outside the first and second boundaries in the known coordinate system, such as using the ray casting technique described above. If outside, then the control system 141 may be configured to set the duration threshold to a first predefined duration, and if between, then the control system 141 may be configured to set the duration threshold to a second predefined duration that is less than the first predefined duration threshold, achieving a similar effect as described above.

In some implementations, the control system 141 may also be configured to determine whether the end effector 240, 340, 440 of a surgical instrument 220, 320, 420 has crossed a virtual boundary associated with a region of interest (e.g., the first virtual boundary mentioned above) or is located in a region of interest, such as using the ray casting technique described above. If so, then the control system 141 may be configured to set the duration threshold to another predefined duration (e.g., a third predefined duration) that differs from the predefined duration to which the duration threshold is set when the end effector 240, 340, 440 is outside the virtual boundary or region of interest (e.g., the second predefined duration described above). In some instances, the another predefined duration may be greater than the previous predefined duration, as once the surgeon enters a region of interest or crosses a virtual boundary associated with a region of interest, it may be assumed that the surgeon is already exercising increased caution and/or receiving notifications of occlusion events via other means (e.g., the LLA indicator 142), and avoiding distracting alerts or interruptions may be desired. For example, assuming the first and second predefined durations described above, the third predefined duration may be greater than the second predefined threshold and/or equal to the first predefined duration corresponding to the surgical instrument 220, 320, 420 being located relatively far from the region of interest.

In a fourth example, responsive to an occlusion event, the control system 141 may be configured to control at least one of the drive motor 245, 345, 445 and the alert module 255, 355, 455 based on a direction of movement of the instrument 220, 320, 420 relative to a region of interest (e.g., towards or away from the region of interest) prior to the occlusion event. The direction of movement may be determined based on a history of poses of the instrument 220, 320, 420 tracked by the navigation system 100 in the known coordinate system relative to the virtual boundaries. The history of poses may include: a first pose of the instrument 220, 320, 420 based on the tracked pose of the instrument relative to the virtual boundary at a first time prior to the occlusion event, and a second pose of the instrument 220, 320, 420 based on the tracked pose of the instrument relative to the virtual boundary at a second time prior to the occlusion event. The control system 141 may be configured to calculate the direction of movement of the instrument 220, 320, 420 by comparing the second pose to the first pose. More specifically, if the first pose indicates that the instrument 220, 320, 420, or more particularly the end effector 240, 340, 440, was 10 mm from the virtual boundary, and the second pose indicates that the instrument 220, 320, 420, or more particularly the end effector 240, 340, 440, was 5 mm from the virtual boundary, the control system can assume that the instrument 220, 320, 420 was moving toward the virtual boundary, and correspondingly the region of interest associated with the virtual boundary, when the occlusion event started. Since the instrument 220, 320, 420 was moving toward the virtual boundary/region of interest as the occlusion event started, it can be assumed that the instrument 220, 320, 420 is still moving toward the virtual boundary/region of interest during the occlusion event. As such, the control system 141 may be configured to alert the surgeon and/or control the speed of the drive motor 245, 345, 445 if the instrument 220, 320, 420 was moving toward the virtual boundary/region of interest when the occlusion event started.

On the other hand, the control system 141 may not alert the surgeon and/or control the drive motor 245, 345, 445 if the instrument 220, 320, 420 was moving away from the virtual boundary/region of interest when the occlusion event started. For example, if the instrument 220, 320, 420 was 5 mm from the virtual boundary during the first pose and 10 mm from the virtual boundary during the second pose, the instrument 220, 320, 420 can be assumed to be moving away from the virtual boundary/region of interest. The surgeon may be moving the tool away from the patient for whatever reason, and it may thus be of relative low value to alert the surgeon that the instrument 220, 320, 420 is occluded from view and/or control the drive motor 245, 345, 445 of the instrument 220, 320, 420, which may no longer be close enough to affect the region of interest.

In some implementations, the control system 141 may be configured to scale the above-described duration threshold, which may have been initially set based on the determined distance as described above, according to the direction of movement of the instrument 220, 320, 420 relative to the region of interest. For instance, the control console 141 may be configured to downscale the set duration threshold by a predefined scale value responsive to determining that the instrument 220, 320, 420 is moving towards the virtual boundary/region of interest, and upscale the set duration threshold by a predefined scale value responsive to determining that the instrument 220, 320, 420 is moving away the virtual boundary/region of interest. In some examples, the upscale and downscale values may be of equal magnitude. The control system 141 may then be configured to compare the scaled duration threshold to the duration of the occlusion event to determine whether to alert the surgeon and/or control the drive motor 245, 345, 445 as described herein.

In a fifth example, in response to an occlusion event, the control system 141 may be configured to control at least one of the drive motor 245, 345, 445 and the alert module 255, 355, 455 based on a velocity of the instrument 220, 320, 420 associated with the occlusion event (e.g., immediately prior to the occlusion event). The velocity may be determined based on a history of poses of the instrument 220, 320, 420 tracked by the navigation system 100 in the known coordinate system relative to the virtual boundaries, such as immediately prior to the occlusion event. The history of poses may include the first and second poses of the instrument as described in the fourth example above. However, instead of taking only the direction of movement of the instrument 220, 320, 420 into account when determining the output of the control system 141, the speed of the instrument 220, 320, 420 may also be considered. Therefore, the control system 141 can understand how far the instrument 220, 320, 420 is expected to move during occlusion events of certain lengths. For example, the control system 141 may be configured to determine, based on the second pose compared to the first pose, that the instrument 220, 320, 420 is moving toward the virtual boundary/region of interest. At this point, the control system 141 may also be configured to calculate the velocity of the instrument 220, 320, 420 based on the difference between the second pose and the first pose divided by the difference between the second time and the first time. This results in a calculation of a speed of the instrument 220, 320, 420 in addition to the direction of the instrument.

In determining whether to control the alert module 255, 355, 455 and/or control the drive motor 245, 345, 445 responsive to an occlusion event, the control system 141 may be configured to consider the velocity of the instrument 220, 320, 420 prior to or during the occlusion event. Compared to a slower moving instrument 220, 320, 420, a faster moving instrument 220, 320, 420 moving closer to a virtual boundary/region of interest may be more likely to unintentionally cross the virtual boundary and/or enter the region of interest associated with the virtual boundary during and/or prior to the occlusion event than a slower moving surgical instrument 220, 320, 420 moving towards a virtual boundary/region of interest or a surgical instrument 220, 320, 420 moving away from the virtual boundary/region of interest. The control system 141 may thus be configured to alert the surgeon and/or control the speed of the drive motor 245, 345, 445 prior to or a short amount of time after onset of the occlusion event responsive to the velocity of the surgical instrument 220, 320, 420 indicating that the surgical instrument 220, 320, 420 is moving towards a virtual boundary/region of interest at a speed greater than a set threshold.

Additionally or alternatively, the control system 141 may be configured to consider the velocity of the instrument 220, 320, 420 when determining the duration threshold of the occlusion event described above. For example, the control system 141 may be configured to set the duration threshold to a relatively low duration (e.g., 0.01 second), and thereby alert the surgeon and/or control the drive motor 245, 345, 445 following a brief (e.g. 0.01 seconds) occlusion event, if the instrument 220, 320, 420 is moving toward the virtual boundary/region of interest at a relatively high speed (e.g., greater than a predefined speed threshold). Conversely, the control system 141 may be configured to set the duration threshold to a relatively high duration (e.g., 0.1 seconds), and thereby alert the surgeon and/or control the drive motor 245, 345, 445 following a longer occlusion event (e.g., 0.1 seconds), if the instrument 220, 320, 420 is moving away from the virtual boundary/region of interest or is moving towards the virtual boundary/region of interest at a relatively slow speed (e.g., less than the predefined speed threshold).

In some examples, when the velocity indicates the surgical instrument 220, 320, 420 is moving towards a virtual boundary/region of the interest, the control system 141 may be configured to set the duration threshold to a shorter duration than when the velocity indicates movement away from the virtual boundary/region of interest. In some examples, the set durations may depend on the magnitude of the velocity. For instance, assuming the velocity indicates movement towards the virtual boundary/region of interest, the control system 141 may be configured to set the duration threshold to shorter durations with increasing speed, such as according to a linear function. Conversely, assuming the velocity indicates movement away from the virtual boundary/region of interest, the control system 141 may be configured to set the duration threshold to longer durations with increasing speed, such according to a linear function.

In some implementations, the control system 141 may be configured to scale the duration threshold, which may have been initially set based on the determined distance as described above, according to the velocity of the instrument 220, 320, 420. For instance, the control console 141 may be configured to downscale the set duration threshold by a given scale value responsive to determining that the instrument 220, 320, 420 is moving towards the virtual boundary/region of interest, and upscale the set duration threshold by a given scale value responsive to determining that the instrument 220, 320, 420 is moving away the virtual boundary/region of interest. The magnitude of the applied upscale and downscale values may be fixed, or alternatively may be determined by the control system 141 based on the magnitude of the velocity of the instrument 220, 320, 420. For instance, the magnitude of the scale value applied by the control system 141 may increase with increasing speed, such as according to a linear function, regardless of whether the duration threshold is being upscaled or downscaled. The control system 141 may then be configured to compare the scaled duration threshold to the duration of the occlusion event to determine whether to alert the surgeon and/or control the drive motor 245, 345, 445 as described herein.

In a sixth example, the control system 141 may be configured to control at least one of the drive motor 245, 345, 445 and the alert module 255, 355, 455 responsive to an occlusion event based on the speed of the instrument 220, 320, 420 prior to the occlusion event. The speed may be determined based on a history of poses of the instrument 220, 320, 420 tracked by the navigation system 100. The history of poses may include: the first pose of the instrument 220, 320, 420 based on the tracked pose of the instrument at the first time prior to the occlusion event, and the second pose of the instrument 220, 320, 420 based on the pose of the instrument at the second time prior to the occlusion event. Since speed, unlike velocity, does not include a directional component, the first and second poses do not need to be calculated relative to the virtual boundary. Alternatively, the speed of the instrument 220, 320, 420 may be calculated by an inertial measurement unit (IMU) (not shown) attached to the instrument 220, 320, 420.

Regardless of the method by which the speed of the instrument 220, 320, 420 is calculated, the control system 141 may then be configured to determine whether to control the alert module 255, 355, 455 and/or control the drive motor 245, 345, 445 responsive to an occlusion event based on the speed of the instrument 220, 320, 420 prior to start of the occlusion event. Similar to the velocity example, the instrument 220, 320, 420 may be more likely to unintentionally cross a virtual boundary and/or enter a region of interest associated with the virtual boundary when it is moving at a faster speed. The control system 141 may thus be configured to control the alert module 255, 355, 455 and/or the drive motor 245, 345, 445 responsive to the speed of the instrument 220, 320, 420 being greater than a predefined speed threshold.

In addition or alternatively, the control system 141 may be configured to determine the duration threshold described above based on the speed of the instrument 220, 320, 420. For example, if the instrument 220, 320, 420 was moving at a relatively low speed prior to the occlusion event (e.g., less than a predefined speed threshold), then the control system 141 may be configured to set the duration threshold to a relatively high duration (e.g., 0.1 seconds), and thereby alert the surgeon and/or control the drive motor 245, 345, 445 following a longer (e.g. 0.1 seconds) occlusion event. However, if the instrument 220, 320, 420 was moving at a relatively high speed prior to the occlusion event (e.g., less than a predefined speed threshold), then the control system 141 may be configured to set the duration threshold to a relatively low duration (e.g., 0.01 seconds), and thereby alert the surgeon and/or control the drive motor 245, 345, 445 following a brief (e.g. 0.01 seconds) occlusion event. In some examples, the duration threshold may be set to fixed relatively high and low durations depending on whether the speed is less than or greater than a predefined speed threshold. Alternatively, the set duration threshold may be set according to a function that defines shorter durations for the duration threshold as a function increasing speeds, such as according to a linear relationship.

In some implementations, the control system 141 may be configured to scale the duration threshold, which may have been initially set based on the determined distance as described above, according to the speed of the instrument 220, 320, 420. For instance, the control system 141 may be configured to downscale the duration threshold based on the speed of the instrument 320, 320, 420 such that, the greater the speed, the greater the scale value applied to the duration threshold. Alternatively, the control system 141 may be configured to upscale and downscale the duration threshold based on the speed of the instrument 320, 320, 420 such that, responsive to determining that the speed is greater than a threshold speed, the duration threshold is downscaled according to a scaled value, which may have a fixed magnitude or a variable magnitude based on the extent of the speed. Conversely, responsive to determining that the speed is less than or equal to the threshold speed, the duration threshold may be upscaled according to a scaled value, which may also have a fixed magnitude or a variable magnitude based on the extent of the speed. The control system 141 may then be configured to compare the scaled duration threshold to the duration of the occlusion event to determine whether to alert the surgeon and/or control the drive motor 245, 345, 445 as described herein.

In a seventh example, the control system 141 may be configured to control at least one of the drive motor 245, 345, 445 and the alert module 255, 355, 455 responsive to an occlusion event based on an acceleration of the instrument 220, 320, 420 associated with the occlusion event. Similar to the direction, velocity, and speed of the instrument 220, 320, 420 described above, the control system 141 may be configured to determine the acceleration based on the history of poses of the instrument 220, 320, 420 tracked by navigation system 100. However, the acceleration may be calculated based on at least the first pose, the second pose, and a third pose of the instrument 220, 320, 420. The third pose of the instrument 220, 320, 420 may be based on the tracked pose of the instrument 220, 320, 420 at a third time prior to the occlusion event. The first, second, and third poses may be determined relative to a virtual boundary or without reference to a virtual boundary. In order to determine the acceleration, a first velocity or a first speed is calculated based on the difference between the second pose and the first pose. After the first velocity or first speed is calculated, a second velocity or second speed is calculated based on the difference between the third pose and the second pose. Finally, the acceleration is calculated based on the either: the difference between the second velocity and the first velocity, or the second speed and the first speed. Using velocity allows the control system 141 to determine a directional component of the acceleration, but this can increase computational costs and so speed may be used instead. Alternatively, the acceleration of the instrument 220, 320, 420 may be calculated by an inertial measurement unit (IMU) attached to the instrument 220, 320, 420.

Regardless of the method by which the acceleration of the instrument 220, 320, 420 is calculated, the control system 141 may be configured to then determine whether to control the alert module 255, 355, 455 and/or control the drive motor 245, 345, 445 responsive to an occlusion event based on the acceleration of the instrument 220, 320, 420 prior to start of the occlusion event. Similar to the velocity and speed examples, the instrument 220, 320, 420 may be more likely to unintentionally cross a virtual boundary and/or enter a region of interest associated with the virtual boundary when it has a higher acceleration. The control system 141 may thus be configured to control the alert module 255, 355, 455 and/or the drive motor 245, 345, 445 responsive to the instrument 220, 320, 420 having had a relatively high acceleration prior to the occlusion event, such as indicated by the acceleration being greater than a predefined acceleration threshold. Further, if the acceleration was calculated using the first, second, and third velocities and therefore includes a directional component, the directional component of the acceleration can be considered by the control system 141. For example, the control system 141 may be configured to determine whether the instrument 220, 320, 420 was accelerating toward the virtual boundary/region of interest prior to the occlusion event. If so, then the control system 141 may be configured to control the alert module 255, 355, 455 and/or the drive motor 245, 345, 445 responsive to the instrument 220, 320, 420 having had a relatively high acceleration prior to the occlusion event, such as indicated by the acceleration being greater than a predefined acceleration threshold. If not, then the control system 141 may be configured to allow the occlusion event to persist without such control based on the assumption that the instrument 220, 320, 420 is being moved away from the patient.

In addition or alternatively, the control system 141 may be configured to determine the duration threshold discussed above based on the acceleration of the instrument 220, 320, 420. For example, the control system 141 may be configured to set the duration threshold to a relatively long duration (e.g., 0.1 seconds), and thereby alert the surgeon and/or control the drive motor 245, 345, 445 following a longer (e.g., 0.1 seconds) occlusion event, if the acceleration is relatively low towards the virtual boundary/region of interest or if the acceleration is away from the virtual boundary/region of interest. Conversely, the control system 141 may be configured to set the duration threshold to a relatively short duration (e.g., 0.01 seconds), and thereby alert the surgeon and/or control the drive motor 245, 345, 445 following a brief (e.g. 0.01 seconds) occlusion event, if the acceleration is relatively high and towards the virtual boundary/region of interest.

In some examples, when the acceleration indicates the surgical instrument 220, 320, 420 is accelerating towards a virtual boundary/region of the interest, the control system 141 may be configured to set the duration threshold to a shorter duration than when the acceleration indicates acceleration away from the virtual boundary/region of interest. In some examples, the duration to which the duration threshold is set may depend on the magnitude of the acceleration. For instance, assuming the acceleration indicates movement towards the virtual boundary/region of interest, the control system 141 may be configured to set the duration threshold to shorter durations with increasing acceleration magnitude, such as according to a linear function. Conversely, assuming the acceleration indicates movement away from the virtual boundary/region of interest, the control system 141 may be configured to set the duration threshold to longer durations with increasing acceleration magnitude, such as according to a linear function.

In some implementations, the control system 141 may be configured to scale the duration threshold, which may have been initially set based on the determined distance as described above, according to the acceleration of the instrument 220, 320, 420. For instance, the control console 141 may be configured to downscale the set duration threshold by a given scale value responsive to determining that the instrument 220, 320, 420 is accelerating towards the virtual boundary/region of interest, and upscale the set duration threshold by a given scale value responsive to determining that the instrument 220, 320, 420 is accelerating away the virtual boundary/region of interest. The magnitude of the applied upscale and downscale values may be fixed, or may be variable based on the magnitude of the acceleration of the of the instrument 220, 320, 420. For instance, the magnitude of the scale value applied by the control system 141 may increase with increasing acceleration magnitude, such as according to a linear function, regardless of whether the duration threshold is being upscaled or downscaled. The control system 141 may then be configured to compare the scaled duration threshold to the duration of the occlusion event to determine whether to alert the surgeon and/or control the drive motor 245, 345, 445 as described herein.

In some implementations, whether the velocity, speed, acceleration associated with an occlusion event is used to determine the duration threshold may be based on the type of end effector 240, 340, 440 or type of surgical instrument 220, 320, 420 currently being operated. To this end, the control system 141 may be configured to determine whether the type of surgical instrument 220, 320, 420 or the type of end effector 240, 340, 440 being operated, which may be indicated via user input. As one example, responsive to determining that the type of end effector 240, 340, 440 is a driver, the control system 141 may be configured to determine (e.g., scale) the duration threshold based on the velocity of the surgical instrument 220, 320, 420 associated with the occlusion event, as described above. Conversely, responsive to determining that the type of end effector 240, 340, 440 is a bur, the control system 141 may be configured to determine (e.g., scale) the duration threshold based on the speed of the surgical instrument 220, 320, 420 associated with the occlusion event, as described above.

In an eighth example, responsive to an occlusion event, the control system 141 may be configured to control at least one of the drive motor 245, 345, 445 and the alert module 255, 355, 455 based on a motion parameter of the instrument 220, 320, 420 prior to the occlusion event. The motion parameter may be determined based on at least one of the direction, velocity, speed, and or acceleration of the instrument 220, 320, 420. The direction, velocity, speed, and or acceleration of the instrument 220, 320, 420 may be determined as described above. Additionally, the surgeon may have the choice to choose the specific parameters which are included in the motion parameter. For example, the graphical user interface 150 may include a selection means by which the surgeon can tell the control system 141 to consider the specified parameters when determining the appropriate response to the occlusion event. The selection means could be a drop-down menu, a series of radio buttons, a series of check boxes, or any suitable alternative.

In a ninth example, the control system 141 may be configured to control at least one of the drive motor 245, 345, 445 and the alert module 255, 355, 455 based on occlusion events involving the patient tracker PT. Since the patient is largely stationary during most medical procedures, the control system 141 may be configured to determine the duration of the occlusion event and respond similarly to the first example. For example, the control system 141 may be configured to determine the duration threshold and ignore any occlusion events where the duration of the occlusion event is shorter than the duration threshold. Further, the control system 141 may be configured to control at least one of the drive motor 245, 345, 445 and the alert module 255, 355, 455 based on the characteristics of the occlusion events involving the patient tracker PT, which may include one or more characteristics of the occlusion event itself and/or of the instrument 220, 320, 420 associated with the occlusion event (e.g., during and/or prior to the occlusion of the patient tracker PT). More specifically, the control system 141 may be configured to determine the pose of the surgical instrument 220, 320, 420 during and/or prior to the occlusion event, the distance between the instrument 220, 320, 420 and the virtual boundaries/regions of interest during and/or prior to the occlusion event, the velocity of the instrument 220, 320, 420 during and/or prior to the occlusion event, the speed of the instrument 220, 320, 420 during and/or prior to the occlusion event, the acceleration of the instrument 220, 320, 420 during and/or prior to the occlusion event, and/or the motion parameter of the instrument 220, 320, 420 during and/or prior to the occlusion of the patient tracker PT. The above characteristics of the instrument 220, 320, 420 may be calculated and used as described in the above examples. For example, the control system 141 may be configured to determine the duration threshold for occlusion events involving the patient tracker PT based on the distance between the instrument 220, 320, 420 and the virtual boundaries/regions of interest during and/or prior to the occlusion event. The duration threshold may be set to a shorter duration when the instrument 220, 320, 420 is closer to the virtual boundaries/regions of interest than when the instrument 220, 320, 420 is farther from the virtual boundaries/regions of interest. Similar combinational examples are contemplated.

It is further contemplated to combine the above examples in any combination. For example, the control system 141 may be configured to control at least one of the drive motor 245, 345, 445 and the alert module 255, 355, 455 based on the duration of the occlusion event, the motion parameter of the instrument 220, 320, 420, and the distance between the instrument 220, 320, 420 and the virtual boundaries. Other combinations are contemplated.

The control system 141 may further control the LLA indicator 142 configured to inform the surgeon of the occurrence of the occlusion event. In general, the LLA indicator 142 may include a visually perceptible device, such as an LED or visual display, which is activated by the control system 141 when occlusion events are detected. The LLA indicator 142 may be included so that the surgeon is aware of occlusion events that are otherwise deemed irrelevant or insignificant pursuant to the above examples. For example, assuming the control system 141 is configured, responsive to an occlusion event, to control at least one of the drive motor 245, 345, 445 and the alert module 255, 355, 455 based on the distance between the instrument 220, 320, 420 and the virtual boundaries during and/or prior to the occlusion event, such control may not occur if the instrument 220, 320, 420 is far away from the patient. However, in the same example, the LLA indicator 142 may be activated even though the drive motor 245, 345, 445 and the alert module 255, 355, 455 are not actively being controlled by the control system 141. The LLA indicator 142 may thus suggest to the surgeon that they should bring the instrument 220, 320, 420 into view of the tracking unit 110 prior to approaching the patient with the instrument 220, 320, 420 Referring to FIG. 6, an exemplary configuration of a surgical system is illustrated including the surgical navigation system 100 and the first surgical instrument 220 described above. While only the first surgical instrument is illustrated in FIG. 6, it is contemplated that any of the surgical instruments 220, 320, 420 described above may be included in the system. The surgical system may also include a plurality of end-effectors 240A, 240B, 240C that are removably couplable to the handpiece 225 of the first surgical instrument 220. The end-effectors 240A, 240B, 240C may also be referred to as end-effectors, surgical attachments, and/or tool attachments. For example, the surgical system may include a first end-effector 240A including a drill for cutting and/or boring a hole in biological material. The surgical system may also include a second end-effector 240B including a tap for creating threads on the interior surface of a hole or aperture. The surgical system may also include a third end-effector 240C including a driver for driving or inserting a screw within the hole or aperture. Each of the end-effectors 240A, 240B, 240C may include an instrument tracking device 230A, 230B, 230C including a unique configuration and/or arrangement of markers 235A, 235B, 235C. The navigation system 100 may be configured to identify the end-effectors 240A, 240B, 240C based on a known association with a particular handpiece 225 and the unique size, shape, and/or arrangement relative to the markers 235A, 235B, 235C of the instrument tracking devices 230A, 230B, 230C that is attached to the specific handpiece 225. The navigation system may then be configured to provide the virtual boundaries (Boundary 1, 2, 3, 4) and/or alert zones (Zones 1, 2, 3, 4) for the appropriate end-effectors 240A, 240B, 240C that are presently being navigated. Exemplary methods of navigating instruments relative to virtual boundaries, alert zones, target trajectories, and/or target depths based on the specific instrument being navigation is described in International Patent Publ. No. 2021/062373, which is incorporated by reference above.

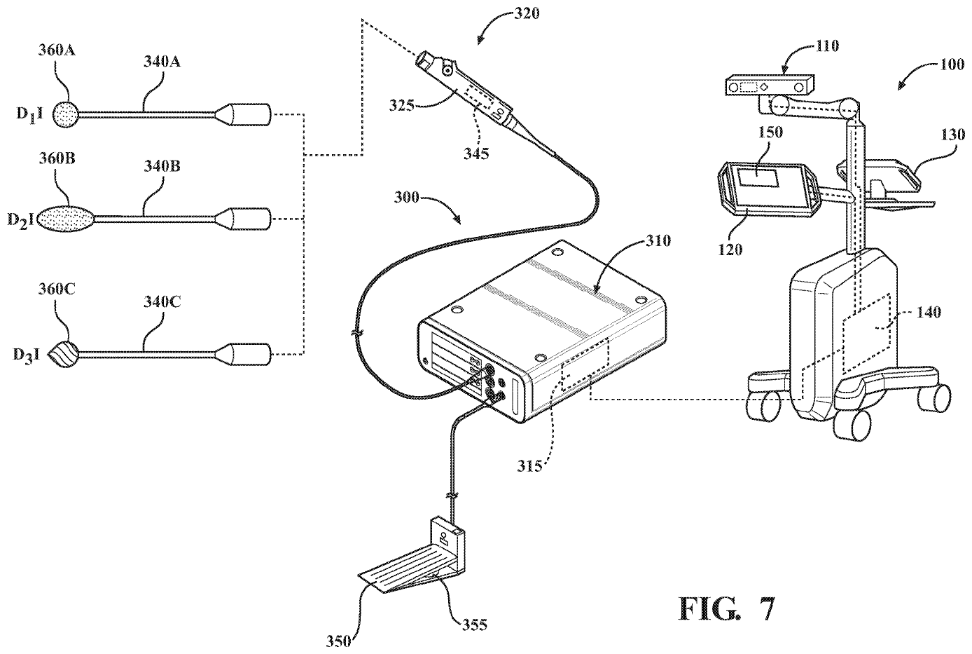

Referring to FIG. 7, an exemplary configuration of a surgical system is illustrated including the surgical navigation system 100 and the second surgical instrument 320 described above. While only the second surgical instrument 320 is illustrated in FIG. 7, it is contemplated that any of the surgical instruments 220, 320, 420 described above may be included in the system. The surgical system may also include a plurality of end-effectors 340A, 340B, 340C that are removably couplable to the handpiece 325 of the second surgical instrument 320. The end-effectors 340A, 340B, 340C may also be referred to as end-affecters, surgical attachments, and/or tool attachments. For example, the surgical system may include a first end-effector 340A including a first bur head 360A having a first diameter head D1. The surgical system may also include a second end-effector 340B including a second bur head 360B having a second diameter head D2. The surgical system may also include a third end-effector 340C including a third bur head 360C having a third diameter head D3. It is also contemplated that the head of each the end-effector 340A, 340B, 340C may vary by shape, material, and/or cutting type. It is also contemplated that the length of the shaft may vary from one end-effector 340A, 340B, 340C to the next. The surgical navigation system 100 may be configured to identify which of the end-effectors 340A, 340B, 340C is coupled to the handpiece 325 by similar methods described above in reference to the end-effectors 240A, 240B, 240C. The navigation system may then be configured to provide the virtual boundaries (Boundary 1, 2, 3, 4) and/or alert zones (Zones 1, 2, 3, 4) for the appropriate end-effectors 340A, 340B, 340C that are presently being navigated. Exemplary methods of navigating instruments relative to virtual boundaries, alert zones, target trajectories, and/or target depths based on the specific instrument being navigation is described in International Patent Publ. No. 2021/062373, which is incorporated by reference above.

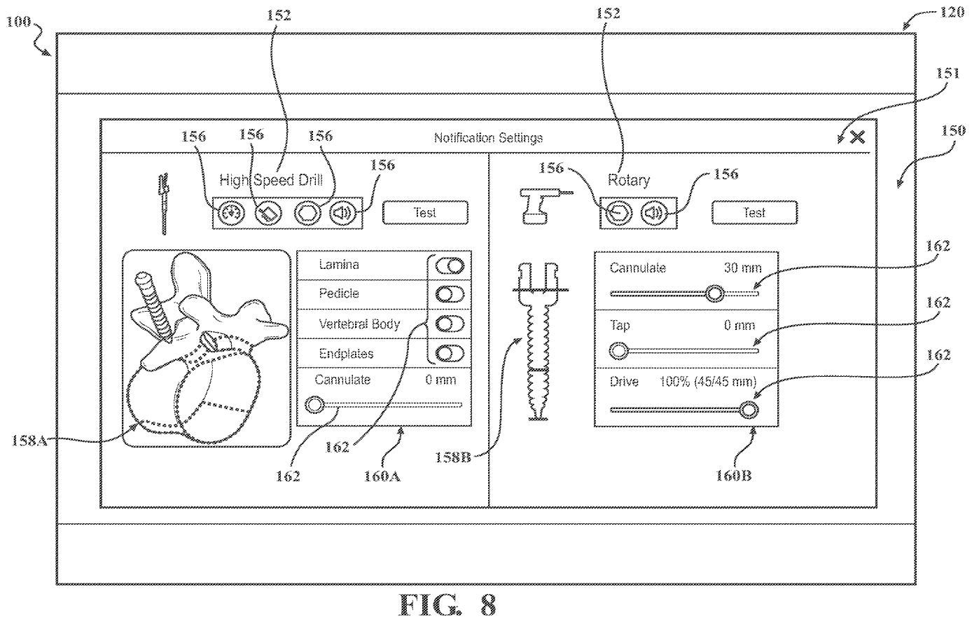

Referring to FIG. 8, an exemplary configuration of the graphical user interface (GUI) 150 of the navigation system 100 is illustrated. The graphical user interface (GUI) 150 may be configured as a touch screen on the display unit 120 of the navigation system 100. As illustrated in FIG. 8, the graphical user interface (GUI) 150 may include a plurality of buttons and/or prompts that are selectable and/or manipulatable by the surgeon. For example, the graphical user interface (GUI) 150 may include an exemplary alert setup interface 151 or window including a number of buttons that are selectable or manipulatable by the user to modify or adjust the various settings for the alerts to be provided during the execution of a medical procedure. The alert setup interface 151 may include at least one tool selection button 152. The tool selection button(s) 152 may allow the surgeon to select the surgical instrument assembly 200, 300, 400 from a populated list of surgical instruments, or may allow the surgeon to input a specific surgical instrument assembly 200, 300, 400 that will be utilized during execution of the surgical procedure. For example, the tool selection button 152 may allow the surgeon to select the second surgical instrument 320 including a high-speed cutting bur. This identifies the particular surgical instrument 320 to the navigation system 100, allowing the navigation system 100 to populate the various virtual boundaries and/or alert zones that will be utilized for the identified instrument. The tool selection button(s) 152 may also be configured to allow the surgeon to select the end-effectors 240, 340, 440 that may be coupled to the surgical instrument 220, 320, 420. The alert setup interface 151 may also include one or more alert buttons 156. The alert buttons 156 may be utilized to manipulate the various alerts described above. For example, the alert buttons 156 may be configured to allow the user to activate or deactivate: an alert related to the rotational speed of the end-effector 240, 340, 440, a tactile alert, a visual alert, and/or an audible alert.

The alert setup interface 151 of the graphical user interface (GUI) 150 may also include one or more alert graphics 158A, 158B. The alert graphic(s) 158A, 158B may be specific to the particular surgical instrument 220, 320, 420 and/or end-effector 240, 340, 440 selected via the GUI 150, and may be configured to provide a schematic and/or visual representation of the location of the various virtual boundaries and/or alert zones. The first alert graphic 158A may include a visual representation of the surgical site, regions of interest in the surgical site, and any implants or devices to be inserted during the medical procedure to assist the surgeon in identifying the location of the procedure and with setting the various alerts. For example, as illustrated in FIG. 8, the first alert graphic 158A includes a visual of the vertebral body with the region where the procedure will take place outlined in dotted lines. The first alert graphic may also include a visual of the pedicel screw to be inserted during the procedure.

The second alert graphic 158B may be configured to provide a visual representation of the implant or device to be inserted during the procedure along with markers indicating the various virtual boundaries (Boundary 1, 2, 3, 4) relative to the implant or device to assist the surgeon in adjusting or modifying the location where the alerts assigned to each of the various virtual boundaries and/or alert zones should be triggered. For example, as illustrated in FIG. 8, the second alert graphic 158B includes a visual representation of the pedicle screw to be inserted and markers along the pedicle screw indicating the location of the various virtual boundaries (Boundary 1, 2, 3, 4) relative to the pedicle screw that will trigger the various alerts during the procedure.

The alert setup interface 151 of the graphical user interface (GUI) 150 may also include one or more virtual boundary setup interface(s) 160A, 160B. The boundary setup interface(s) 160A, 160B may include one more prompts or buttons 162 for setting the virtual boundaries and/or manipulating when the virtual boundaries, alert zones, and/or target depths will trigger one or more of the various alerts described above. This allows the navigation system 100 to determine which and how many virtual boundaries, alert zones, and/or target depths to provide. For example, if the surgeon manipulates the first button 162 to indicate a Laminotomy will be performed, the navigation system 100 will understand that this is a resection process and the navigation system 100 will know to identify and provide the various alert zones around the critical structures of the vertebra to assist the surgeon in executing the procedure.

An exemplary graphical user interface is described in International Patent Publ. No. 2021/062373, which is incorporated by reference above.

Referring to FIG. 9, the exemplary graphical user interface (GUI) 150 including an occlusion event button 170 and an occlusion event interface 172 is illustrated. The occlusion event button 170 is configured to present the occlusion event interface 172 to the user, and the occlusion event interface 172 is configured to allow the user to configure the control system 141. More specifically, the occlusion event interface 172 may include selection means to allow the user to select the characteristics of occlusion event to be considered by the control system 141. In response to the selected characteristics, the control system 141 may control the alert module 255, 355, 455 and/or the drive motor 245, 345, 445 of the surgical instrument 220, 320, 420 responsive to an occlusion event based on the selected characteristics. The occlusion event interface 172 may also allow the user to configure the control system 141 to control the alert module 255, 355, 455 and/or the drive motor 245, 345, 445 based on the occlusion of the patient tracker PT.

The occlusion event interface 172 may be utilized by the user to configure the control system 141 based on which medical procedure is being performed and/or which surgeon is performing the procedure so as to fit the needs and/or preferences of the surgeon. In particular, the occlusion event interface 172 may include selection means to select at least one of the following characteristics: a cause of the occlusion event, a duration of the occlusion event, a pose of the surgical instrument 220, 320, 420, such as relative to the virtual boundaries/regions of interest, a distance between the instrument 220, 320, 420 and the virtual boundaries/regions of interest, a velocity of the instrument 220, 320, 420, a speed of the instrument 220, 320, 420, an acceleration of the instrument 220, 320, 420, and/or a motion parameter of the instrument 220, 320, 420 during and/or prior to the occlusion event. Other characteristics, including some included above and not listed here, are also contemplated. For example, the surgeon may prefer the control system 141 to control the alert module 255, 355, 455 and/or the drive motor 245, 345, 445 of the surgical instrument 220, 320, 420 in response to any occlusion events lasting longer than 0.1 seconds. In such an example, the occlusion event interface 172 may include time selection means to choose the duration threshold. In addition, the occlusion event interface 172 may include alert selection means to allow the user/surgeon to choose how the control system 141 controls the alert module 255, 355, 455 and/or the drive motor 245, 345, 445 of the surgical instrument 220, 320, 420 in response to the occlusion events above a chosen duration threshold. For example, the surgeon may prefer the control system 141 to give an audible alert with the alert module 255, 355, 455 and to slow down the drive motor 245, 345, 445 in response to the occlusion events.

The occlusion event interface 172 may also include a button (not shown) configured to switch the occlusion detection aspect of the control system 141 between an enabled state and a disabled state.

Still referring to FIG. 9, an exemplary loss-of-localization-awareness (LLA) indicator 142 is also illustrated. As described above, the LLA indicator 142 may include a software routine comprising instructions stored in memory accessible by the control system 141, the surgical navigation system 100, one or more of the surgical instrument assemblies 200, 300, 400, and/or other elements of the surgical system 10. The LLA indicator 142 is configured to display an indication of an ongoing occlusion event. As will be appreciated from FIG. 9, the LLA indicator 142 may be realized as a small window off to the side of the GUI 150. In the illustrated example, the LLA indicator 142 is small and off-center so as not to cover too much of the GUI 150. Generally, the LLA indicator 142 includes an indication of whether an occlusion event is being detected. For example, the LLA indicator 142 may be a solid-colored box that switches between two colors to indicate whether an occlusion event is being detected. In such an example, the LLA indicator 142 may be a green box if no occlusion event is detected, and the LLA indicator 142 may change into a red box in response to a detected occlusion event. In another example, the LLA indicator 142 may be a pop-up indicator that is only present when an occlusion event is detected. Other examples are also contemplated.

The LLA indicator 142 may further include means to change the behavior of the LLA indicator 142. In one example, the LLA indicator 142 may include a button configured to switch the LLA indicator 142 between an enabled state in which the LLA indicator 142 displays the occurrence of occlusion events, and a disabled state in which the LLA indicator 142 does not display the occurrence of occlusion events. In another example, the LLA indicator 142 may include a snooze button configured to disable the LLA indicator 142 for a predefined period. In another example, the LLA indicator 142 may include selection means configured to change how the GUI 150 displays the LLA indicator 142. For example, the selection means may allow the user to cause the GUI 150 to display the LLA indicator 142 as a least one of: a pop-up, a stationary box containing a color indication, and other suitable alternatives. In another example, the selection means may allow the user to change the size and/or location of the LLA indicator 142 as displayed by the GUI 150. Combinational examples are also contemplated.

The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the examples is described above as having certain features, any one or more of those features described with respect to any example of the disclosure can be implemented in and/or combined with features of any of the other examples, even if that combination is not explicitly described. In other words, the described examples are not mutually exclusive, and permutations of one or more examples with one another remain within the scope of this disclosure.

Spatial and functional relationships between elements (for example, between controllers, circuit elements, semiconductor layers, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements.

As used herein, the phrase at least one of A, B, and C should be construed to mean a logical (A OR B OR C), using a non-exclusive logical OR, and should not be construed to mean “at least one of A, at least one of B, and at least one of C.” The term subset does not necessarily require a proper subset. In other words, a first subset of a first set may be coextensive with (equal to) the first set.

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “controller” or “module” may be replaced with the term “circuit.” The term “controller” may refer to, be part of, or include: an Application Specific Integrated Circuit (ASIC); a programmable system on a chip (PSoC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The controller may include one or more interface circuits with one or more transceivers. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are the BLUETOOTH wireless networking standard from the Bluetooth Special Interest Group and IEEE Standard 802.15.4.

The controller may communicate with other controllers using the interface circuit(s). Although the controller may be depicted in the present disclosure as logically communicating directly with other controllers, in various implementations the controller may actually communicate via a communications system. The communications system may include physical and/or virtual networking equipment such as hubs, switches, routers, gateways and transceivers. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the controller may be distributed among multiple controllers that are connected via the communications system. For example, multiple controllers may implement the same functionality distributed by a load balancing system. In a further example, the functionality of the controller may be split between a server (also known as remote, or cloud) controller and a client (or user) controller.

Some or all hardware features of a controller may be defined using a language for hardware description, such as IEEE Standard 1364-2005 (commonly called “Verilog”) and IEEE Standard 1076-2008 (commonly called “VHDL”). The hardware description language may be used to manufacture and/or program a hardware circuit. In some implementations, some or all features of a controller may be defined by a language, such as IEEE 1666-2005 (commonly called “SystemC”), that encompasses both code, as described below, and hardware description.

The term code, as used above, may include software, firmware, and/or microcode, and may refer to programs, routines, functions, classes, data structures, and/or objects. The term shared processor circuit encompasses a single processor circuit that executes some or all code from multiple controllers. The term group processor circuit encompasses a processor circuit that, in combination with additional processor circuits, executes some or all code from one or more controllers. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple controllers. The term group memory circuit encompasses a memory circuit that, in combination with additional memories, stores some or all code from one or more controllers.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks and flowchart elements described above may serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

Some examples are described in reference to the following numbered clauses, with specific features laid out in dependent clauses:
1. A method for determining an occlusion event for a tracking device coupled to a hand-held instrument including a drive motor, the method comprising:
tracking the tracking device coupled to the hand-held instrument with a tracking unit;
determining a pose of the instrument;
determining a boundary;
determining if the occlusion event has occurred for the tracking device;
controlling at least one of the alert module and the drive motor based on the occlusion event, the boundary, and the pose of the instrument.
2. A navigation system for tracking a tracking device coupled to a hand-held instrument including a drive motor, the navigation system comprising:
a tracking unit;
an alert module; and
a control system in communication with the tracking unit and the alert module, the control system configured to:
   determine a pose of the instrument,
   determine a boundary,
   control the alert module based on the boundary and the pose of the instrument,
   determine that an occlusion event has occurred for the tracking device, and
   control the drive motor based on the occlusion event, the boundary, and the pose of the instrument.
3. A method for determining an occlusion event for a tracking device coupled to a hand-held instrument including a drive motor, the method comprising:
tracking a pose of the instrument in the known coordinate system with a tracking unit,
determining a boundary in the known coordinate system, the boundary being associated with a region of interest in a surgical procedure,
controlling the alert module based on the boundary and the tracked pose of the instrument in the known coordinate system,
determining that an occlusion event has occurred for the tracking device, and
controlling the drive motor based on the occlusion event, the boundary, and the tracked pose of the instrument.
4. A navigation system for tracking an instrument configured to be held in a hand of a user, the instrument including a drive motor, the system comprising:
a tracking unit configured to cooperate with a tracking device coupled to the instrument to generate tracking data indicative of a pose of the instrument in a known coordinate system; and
a control system in communication with the tracking unit, an alert module for indicating a position of the instrument relative to a region of interest in a surgical procedure, and the instrument, the control system configured to:
   track the pose of the instrument in the known coordinate system based on the tracking data,
   determine a boundary in the known coordinate system, the boundary being associated with the region of interest,
   control the alert module based on the boundary and the tracked pose of the instrument in the known coordinate system,
   determine that an occlusion event has occurred for the tracking device,
   determine a duration of the occlusion event, and
   control the drive motor based on the duration of the occlusion event.
5. The navigation system of clause 4, wherein the control system is configured to:
determine a duration threshold based on one or more characteristics of the occlusion event; and
control the drive motor based on the duration of the occlusion event and the duration threshold.
6. A method for determining an occlusion event for a tracking device coupled to a hand-held instrument including a drive motor, the method comprising:
tracking a pose of the instrument in the known coordinate system with a tracking unit,
determining a boundary in the known coordinate system, the boundary being associated with a region of interest in a surgical procedure,
controlling the alert module based on the boundary and the tracked pose of the instrument in the known coordinate system,
determining that an occlusion event has occurred for the tracking device,
determining a distance between the instrument and the region of interest based on the boundary and the tracked pose of the instrument in the known coordinate system, and
controlling the drive motor based on the distance between the instrument and the region of interest and the occlusion event.
7. A method for determining an occlusion event for a tracking device coupled to a hand-held instrument including a drive motor, the method comprising:
tracking a pose of the instrument in the known coordinate system with a tracking unit,
determining a boundary in the known coordinate system, the boundary being associated with a region of interest in a surgical procedure,
controlling the alert module based on the boundary and the tracked pose of the instrument in the known coordinate system,
determining that an occlusion event has occurred for the tracking device,
determining a duration of the occlusion event,
determining a distance between the instrument and the region of interest based on the boundary and the tracked pose of the instrument in the known coordinate system, and
controlling the alert module based on the duration of the occlusion event and the distance between the instrument and the region of interest.
8. A hand-held surgical system comprising: an instrument configured to be held in a hand of a user, the instrument including a drive motor;
a console coupled to the instrument;
a tracking device coupled to the instrument;
a tracking unit;
an alert module;
a control system in communication with the tracking unit, the alert module, and the console, the control system configured to:
   determine a pose of the instrument,
   determine a boundary,
   control the alert module based on the boundary and the pose of the instrument,
   determine that an occlusion event has occurred for the tracking device, and
   control the drive motor based on the occlusion event, the boundary, and the pose of the instrument.
9. The hand-held surgical system of clause 8, wherein the control system is further configured to:
determine a duration of the occlusion event, and
control the drive motor based on the duration of the occlusion event.
10. The hand-held surgical system of clause 8 or 9, wherein control system is further configured to:
determine a distance between the instrument and the boundary based on the boundary and the pose of the instrument, and
control the drive motor based on the distance and the occlusion event.
11. The hand-held surgical system of any preceding clause, wherein the control system is further configured to:
determine a duration of the occlusion event,
determine a distance between the instrument and the boundary based on the boundary and the pose of the instrument, and
control the drive motor based on the duration, the distance, and the occlusion event.
12. The hand-held surgical system of any preceding clause, wherein the alert module is a vibratable footswitch, a graphical user interface, or a speaker.
13. The hand-held surgical system of any preceding clause, wherein the alert module is defined as a software routine.
14. The hand-held surgical system of clause 13, wherein the software routine is configured to set the drive speed of the motor.

15. The hand-held surgical system of clause 13, wherein the software routine is configured to stop the drive motor.

16. The hand-held surgical system of any preceding clause, the system further comprising a switch in communication with the instrument and the control system, the switch configured to generate an input signal sufficient to control the instrument, and
wherein the control system is further configured to reactivate the drive motor based on the input signal.
17. The hand-held surgical system of any preceding clause, wherein the control system is further configured to:
determine a duration of the occlusion event,
determine a duration threshold, and
control the alert module based on the duration and the duration threshold.
18. The hand-held surgical system of any preceding clause, the system further comprising a switch in communication with the instrument and the control system, the switch configured to generate an input signal sufficient to control the instrument.
19. The hand-held surgical system of any preceding clause, wherein the control system is further configured to determine a duration of the occlusion event, determine a distance between the instrument and the boundary, and
wherein controlling the alert module based on the boundary and the pose of the instrument further includes controlling the alert module based on the duration of the occlusion event and the distance.
20. The hand-held surgical system of any preceding clause, wherein the control system is further configured to:
determine a direction of movement of the instrument based on the pose of the instrument and the boundary, and
control the drive motor based on the direction of movement of the instrument and the occlusion event.
21. The hand-held surgical system of clause 20, wherein the control system is further configured to:
determine a first pose of the instrument based on the pose of the instrument at a first time,
determine a second pose of the instrument based on the pose of the instrument at a second time, and
determine the direction of the instrument based on the first pose of the instrument and the second pose of the instrument.
22. The hand-held surgical system of any preceding clause, wherein the instrument further comprises a battery in electrical communication with the drive motor, and controlling the drive motor includes controlling the battery.
23. The hand-held surgical system of any preceding clause, wherein the control system is further configured to:
determine a velocity of the instrument based on the boundary and the pose of the instrument, and
control the drive motor based on the velocity of the instrument.
24. The hand-held surgical system of clause 23, wherein the control system if further configured to:
determine a first pose of the instrument based on the pose of the instrument at a first time,
determine a second pose of the instrument based on the pose of the instrument at a second time, and
determine the velocity of the instrument based on the first pose of the instrument and the second pose of the instrument.
25. The hand-held surgical system of any preceding clause, wherein the control system is further configured to:
determine a motion parameter of the instrument based on poses of the instrument and boundaries.
26. The hand-held surgical system of clause 25, wherein the motion parameter is selected from at least one of a direction of the instrument, a velocity of the instrument, and an acceleration of the instrument.
27. The hand-held surgical system of any preceding clause, further comprising a localization awareness indicator in communication with the control system.
28. The hand-held surgical system of clause 27, wherein the control system controls the localization awareness indicator based on the occlusion event.
29. The hand-held surgical system of clause 28, wherein the localization awareness indicator is defined as at least one of an auditory, visual, and tactile indicator.
30. The hand-held surgical system of clause 28, wherein the localization awareness indicator is defined as a software routine.
31. The hand-held surgical system of any preceding clause, wherein the boundary is a mesh.
32. The hand-held surgical system of clause 31, wherein the mesh is derived from a patient image.
33. The hand-held surgical system of clause 32, wherein the mesh is derived from a segmentation of the patient image.
34. The hand-held surgical system of any preceding clause, further comprising a patient tracker coupled to a patient.
35. The hand-held surgical system of clause 34, wherein the control system is further configured to:
determine a pose of at least a portion of the patient, and
determine that the occlusion event has occurred for the patient tracker.
36. A hand-held surgical system comprising:
an instrument configured to be held in a hand of a user, the instrument including a drive motor;
a tracking device coupled to the instrument;
a tracking unit;
an alert module;
a control system in communication with the tracking unit and the alert module, the control system configured to:
   determine a pose of the instrument,
   determine a boundary,
   control the alert module based on the boundary and the pose of the instrument,
   determine that an occlusion event has occurred for the tracking device,
   determine a duration of the occlusion event,
   determine a distance between the instrument and the boundary, control the alert module based on the occlusion event, the duration of the occlusion event, the boundary, the pose of the instrument, and the distance between the instrument and the boundary.
37. A hand-held surgical system comprising:
an instrument configured to be held in a hand of a user, the instrument including a drive motor;
a console coupled to the instrument;
a tracking device coupled to the instrument;
a tracking unit;
an alert module;
a control system in communication with the tracking unit, the alert module, and the console, the control system configured to:
   determine a pose of the instrument,
   determine a boundary,
   control the alert module based on the boundary and the pose of the instrument,
   determine that an occlusion event has occurred for the tracking device, and
   determine a distance between the instrument and the boundary based on the boundary and the pose of the instrument
   control the drive motor based on the distance and the occlusion event.
38. The hand-held surgical system of clause 37, wherein the control system determines a distance threshold and is configured to control the drive motor when an occlusion event has occurred and the distance between the instrument and the boundary is less than the distance threshold.
39. The hand-held surgical system of clause 37 or 38, wherein the instrument includes an end effector and the distance between the instrument and the boundary is determined by a length of a line between the end effector and a point on the boundary that is closest to the end effector.
40. The hand-held surgical system of clause 39, where the line is normal to the boundary and crosses the point on the boundary that is closest to the end effector.
41. The hand-held surgical system of clause 39 or 40, wherein the end effector is a burr including a burr head and the line is between the center of the burr head and the boundary.
42. The hand-held surgical system of clause 39 or 40, wherein the end effector is a drill bit including a tip and the line is between the tip of the drill bit and the boundary.
43. The hand-held surgical system of clause 41 or 42, wherein the control system is further configured to determine a direction of the distance relative to the boundary by utilizing ray casting.
44. The hand-held surgical system of clause 43, wherein:
the ray casting includes casting a ray from the center of the burr and into a shaft of the burr; and
the control system determines that the center of the burr has advanced past the boundary if the ray intersects the boundary.
45. The hand-held surgical system of clause 43, wherein:
the ray casting includes casting a ray from the tip of the drill bit and into a remainder of the drill bit; and
the control system determines that the center of the burr has advanced past the boundary if the ray intersects the boundary.
46. The hand-held surgical system of clause 39, wherein the end effector is a driver including a tip and the line is between the tip of the driver and a center of a planned position of a screw.
47. The hand-held surgical system of clause 39, wherein the end effector is a screw including a tip and the line is between the tip of the screw and a center of a planned position of a screw.
48. The hand-held surgical system of clause 46 or 47, wherein:
the boundary is realized a spherical virtual boundary centered on and surrounding a planned screw position, the planned screw position having a length;
the spherical virtual boundary has a diameter equal to the length of the planned screw position; and
the line is between the tip of the screw and the center of the spherical virtual boundary.
49. The hand-held surgical system of clause 48, wherein the control system determines a distance threshold and is configured to control the drive motor when an occlusion event has occurred and the distance is less than the distance threshold.
50. The hand-held surgical system of clause 48, wherein:
the control system determines an adjusted distance between the tip of the screw and an edge of the spherical virtual boundary as a length of the line minus a radius of the spherical virtual boundary, and
the control system determines a distance threshold and is configured to control the drive motor when an occlusion event has occurred and the adjusted distance is less than the distance threshold.
51. The hand-held surgical system of clause 48, wherein:
the planned screw position is realized as a first planned screw position;
the spherical virtual boundary is realized as a first spherical virtual boundary centered on and surrounding the first planned screw position;
the line is realized as a first line between the tip of the screw and the center of the first spherical virtual boundary;
the boundary further includes a second spherical boundary centered on and surrounding a second planned screw position;
the second spherical boundary has a diameter equal to the length of the second planned screw position;
a second line is determined between the tip of the screw and the center of the second spherical virtual boundary; and
the distance between the instrument and the boundary is determined by the shorter of the first line and the second line.
52. The hand-held surgical system of clause 51, wherein the control system determines a distance threshold and is configured to control the drive motor when an occlusion event has occurred and the distance is less than the distance threshold.
53. The hand-held surgical system of clause 51, wherein:
the control system determines an adjusted distance between the tip of the screw and an edge of the spherical virtual boundary as a length of the shorter line minus a radius of the respective spherical virtual boundary; and
the control system determines a distance threshold and is configured to control the drive motor when an occlusion event has occurred and the adjusted distance is less than the distance threshold.
54. The hand-held surgical system of any one of clauses 37-53, wherein the control system is further configured to:
determine a duration of the occlusion event,
control the drive motor based on the duration, the distance, and the occlusion event.
55. The hand-held surgical system of clause 54, wherein the control system determines a duration threshold and the control system is configured to control the drive motor when the duration of the occlusion event is longer than the duration threshold.
56. The hand-held surgical system of clause 55, wherein the duration threshold is based on the distance.
57. The hand-held surgical system of clause 56, wherein:
the duration threshold is realized as a first duration threshold when the distance indicates that the instrument is outside of the boundary and a second duration threshold when the distance indicates that the instrument has crossed the boundary; and
the second duration threshold is shorter than the first duration threshold.
58. The hand-held surgical system of clause 56, wherein:
the duration threshold is realized as a first duration threshold when the distance indicates that the instrument is within a distance threshold of the boundary and a second duration threshold when the distance indicates that the instrument is not within a distance threshold of the boundary; and
the second duration threshold is longer than the first duration threshold.
59. The hand-held surgical system of any one of clauses 37-58, further comprising a localization awareness indicator in communication with the control system.
60. The hand-held surgical system of clause 59, wherein the control system controls the localization awareness indicator based on the occlusion event.

1. A hand-held surgical system comprising:
an instrument configured to be held in a hand of a user, the instrument including a drive motor;
a tracking device coupled to the instrument;
a tracking unit configured to cooperate with the tracking device to generate tracking data indicative of a pose of the instrument in a known coordinate system;
an alert module for indicating a position of the instrument relative to a region of interest in a surgical procedure; and
a control system in communication with the tracking unit, the alert module, and the instrument, the control system configured to:
track the pose of the instrument in the known coordinate system based on the tracking data,
determine a boundary in the known coordinate system, the boundary being associated with the region of interest,
control the alert module based on the boundary and the tracked pose of the instrument in the known coordinate system,
determine that an occlusion event has occurred for the tracking device, and
disable the drive motor based on the occlusion event, the boundary, and the tracked pose of the instrument.

2. The hand-held surgical system of claim 1, wherein the control system is further configured to:
determine a duration of the occlusion event, and
disable the drive motor based on the duration of the occlusion event, the boundary, and the tracked pose of the instrument.

3. The hand-held surgical system of claim 2, wherein the control system is further configured to:
determine a duration threshold based on the boundary and the tracked pose of the instrument; and
disable the drive motor based on the duration of the occlusion event and the duration threshold.

4. The hand-held surgical system of claim 3, wherein the boundary is defined as a first boundary, and the control system is further configured to:
determine a second boundary in the known coordinate system, the second boundary being located farther from the region of interest than the first boundary in the known coordinate system;
determine a position of an end effector of the instrument relative to the first and second boundaries in the known coordinate system based on the tracked pose of the instrument;
responsive to determining that the end effector is outside the first and second boundaries in the known coordinate system, set the duration threshold to a first duration; and
responsive to determining that the end effector is between the first and second boundaries in the known coordinate system, set the duration threshold to a second duration, the second duration being less than the first duration.

5. The hand-held surgical system of claim 3, wherein the control system is further configured to:
determine a positional relationship between the instrument and the region of interest based on the boundary and the tracked pose of the instrument in the known coordinate system; and
determine the duration threshold based on the positional relationship.

6. The hand-held surgical system of claim 3, wherein control system is further configured to:
determine a distance between the instrument and the region of interest based on the boundary and the tracked pose of the instrument in the known coordinate system; and
determine the duration threshold based on the distance.

7. The hand-held surgical system of claim 6, wherein the instrument includes an end effector and the control system is configured to determine the distance between the instrument and the region of interest by being configured to determine a distance between the end effector and the boundary in the known coordinate system.

8. The hand-held surgical system of claim 7, wherein the control system is configured to determine the distance between the end effector and the boundary in the known coordinate system based on a length of a line between the end effector and a point on the boundary that is closest to the end effector in the known coordinate system.

9. (canceled)

10. The hand-held surgical system of claim 8, wherein the end effector is a burr including a burr head and the line is between a center of the burr head and the boundary.

11. The hand-held surgical system of claim 8, wherein the end effector is a drill bit including a tip and the line is between the tip of the drill bit and the boundary.

12. The hand-held surgical system of claim 7, wherein the end effector is a driver including a tip and the control system is configured to determine the distance between the end effector and the boundary based on a length of a line between the tip of the driver and a center of a planned position of a screw in the known coordinate system.

13. The hand-held surgical system of claim 7, wherein the end effector is a screw including a tip and the control system is configured to determine the distance between the end effector and the boundary based on a length of a line between the tip of the screw and a center of a planned position of the screw in the known coordinate system.

14. The hand-held surgical system of claim 12, wherein:
the boundary is realized as a spherical virtual boundary centered on and surrounding the planned screw position, the planned screw position having a length;
the spherical virtual boundary has a diameter equal to the length of the planned screw position; and
the line is between the tip of the screw and a center of the spherical virtual boundary.

15. The hand-held surgical system of claim 14, wherein the control system is configured to:
determine an adjusted distance between the tip of the screw and an edge of the spherical virtual boundary as the length of the line minus a radius of the spherical virtual boundary; and
determine the distance between the end effector and the boundary in the known coordinate system based on the adjusted distance.

16. The hand-held surgical system of claim 12, wherein the planned screw position is defined as a first planned screw position, the spherical virtual boundary is defined as a first spherical virtual boundary centered on and surrounding the first planned screw position, the line is defined as a first line, the control system is configured to:
determine a second spherical virtual boundary in the known coordinate system centered on and surrounding a second planned screw position, the second spherical virtual boundary having a diameter equal to the length of the second planned screw position;
determine a second line between the end effector and the center of the second spherical virtual boundary;
determine that the length of the first line is less than the length of the second line; and
determine the distance between the end effector and the first spherical virtual boundary in the known coordinate system responsive to determining that the length of the first line is less than the length of the second line.

17. The hand-held surgical system of claim 6, wherein the control system is further configured to:
determine whether the distance between the instrument and the region of interest is greater than a threshold distance; and
determine the duration threshold based on the determination of whether the distance between the instrument and the region of interest is greater than the threshold distance.

18. The hand-held surgical system of claim 17, wherein the control system is further configured to:
responsive to determining that the distance between the instrument and the region of interest is greater than the threshold distance, set the duration threshold to a first duration; and
responsive to determining that the distance between the instrument and the region of interest is less than the threshold distance, set the duration threshold to a second duration, the second duration being less than the first duration.

19. The hand-held surgical system of claim 4, wherein the control system is further configured to:
determine whether an end effector of the instrument is positioned in the region of interest based on the boundary and the tracked pose of the instrument in the known coordinate system; and
responsive to determining that the end effector of the instrument is positioned in the region of interest, set the duration threshold to a third duration, the third duration being greater than the second duration.

20-64. (canceled)

65. A navigation system for use with an instrument configured to be held in a hand of a user and an alert module for indicating a position of the instrument relative to a region of interest in a surgical procedure, the instrument including a drive motor, the system comprising:
a tracking unit configured to cooperate with a tracking device coupled to the instrument to generate tracking data indicative of a pose of the instrument in a known coordinate system; and
a control system in communication with the tracking unit, the alert module, and the instrument, the control system configured to:
track the pose of the instrument in the known coordinate system based on the tracking data,
determine a boundary in the known coordinate system, the boundary being associated with the region of interest,
control the alert module based on the boundary and the tracked pose of the instrument in the known coordinate system,
determine that an occlusion event has occurred for the tracking device, and
disable the drive motor based on the occlusion event, the boundary, and the tracked pose of the instrument.

66. A method for navigating a surgical instrument configured to be held in a hand of a user and including a drive motor, the method comprising::
tracking, by a control system in communication with the instrument, a tracking unit, and an alert module for indicating a position of the instrument relative to a region of interest in a surgical procedure, a pose of the instrument in a known coordinate system based on tracking data that is generated by the tracking unit cooperating with a tracking device coupled to the instrument;
determining, by the control system, a boundary in the known coordinate system, the boundary being associated with a region of interest in a surgical procedure;
controlling, by the control system, the alert module based on the boundary and the tracked pose of the instrument in the known coordinate system;
determining, by the control system, that an occlusion event has occurred for the tracking device; and
disabling, by the control system, the drive motor based on the occlusion event, the boundary, and the tracked pose of the instrument.
